(19) United States
(12) Patent Application Publication
(10) Pub. No.: US 2026/0000824 A1
(43) Pub. Date: Jan. 1, 2026

(54) AIRWAY FOR A NEGATIVE PRESSURE WOUND DRESSING

(71) Applicant: ConvaTec Limited, Flintshire (GB)

(72) Inventor: Manjunath Penagondla, Warrington (GB)

(21) Appl. No.: 19/318,581

(22) Filed: Sep. 4, 2025

Related U.S. Application Data
(63) Continuation of application No. PCT/GB2024/050589, filed on Mar. 6, 2024.
(60) Provisional application No. 63/450,476, filed on Mar. 7, 2023.
(60) Provisional application No. 63/514,642, filed on Jul. 20, 2023.
(60) Provisional application No. 63/518,347, filed on Aug. 9, 2023.

(30) Foreign Application Priority Data
Mar. 22, 2023 (GB) 2304150.2
Sep. 12, 2023 (GB) 2313861.3
Sep. 12, 2023 (GB) 2313869.6

(51) Int. Cl. A61M 1/00

(52) U.S. Cl. CPC A61M 1/913; A61M 2207/00

(57) ABSTRACT

An airway for a negative pressure wound dressing, the airway comprising a first strip of material having a first surface comprising at least one groove, the airway further comprising a film arranged over the first surface of the first strip of material and adhered to the first strip of material around its longitudinal edges, wherein the at least one groove is provided with at least one projection to form at least two channels between the first strip and the film, wherein the airway further comprises a second strip of material having a first surface comprising at least one groove provided with at least one projection, the first surface of the second strip of material being arranged over the film and adhered to the film around its longitudinal edges to form at least two channels, between the second strip and the film.

### TECHNICAL FIELD OF THE INVENTION

The present invention relates to an airway for a wound dressing. In particular, the invention concerns an airway for a negative pressure wound dressing.

### BACKGROUND TO THE INVENTION

Wound dressings are known and are generally suitable for treating a variety of wounds, including chronic and acute wound types, such as infected wounds, venous ulcers, diabetic ulcers, burns and surgical wounds.

Negative pressure has been used to treat a range of chronic and acute wounds. Negative pressure may facilitate wound healing through a number of mechanisms, including removal of excess exudate, reduction in periwound edema and increased perfusion. Combined with the physical forces exerted by the negative pressure which draw the wound edges together, this can result in improved wound outcomes. Existing wound dressing systems typically rely on an airway to draw air away from the wound site via a pump.

Existing wound dressing systems often include rigid dressing and connection components, adversely affecting system utility and user comfort. In particular, existing systems use an airway formed of a hollow tube made of a firm plastic material. However, such an arrangement is considerably uncomfortable for the patient if the patient lies on the tube. Conversely, a hollow tube formed of a relatively soft plastic material would be prone to compression if the patient rests upon the tube. Compression of the tube would significantly or completely block the flow of air through the airway, therefore, inhibiting or even preventing wound healing. To overcome this, some systems use a spacer material. The spacer material typically has a three-dimensional structure to aid in wicking fluid or transmitting negative pressure. Conventionally, spacer materials are selected for their ability to channel wound exudate away from a wound and for transmitting negative pressure and/or vented air to the wound site. Despite the presence of the spacer material, known airways comprising spacer materials disadvantageously collapse under the application of negative pressure (typical pressures used in negative pressure wound therapy are between 40 and 150 mmHg). In such cases, the airway becomes obstructed and the flow of air from the wound site is inhibited. To prevent the known airways comprising spacer materials collapsing under the application of negative pressure and in an attempt to keep the airway open and capable of communicating negative pressure to the wound site, it is typical that several layers of spacer fabric are used. However, this often results in an airway comprising a spacer fabric having a thickness of several millimetres and in some cases around 1 cm. Such airways can be uncomfortable for a patient, especially when the airway is lent upon and rubs against the patient's skin. This may cause pressure ulcers and other complications which can result from a wound dressing system being pressed against the skin of a patient.

Such airways comprising a spacer material as described above are relatively firm and inflexible, and this, in close proximity to the wound site, can adversely affect the wound healing process. The airway may become obstructed by movement of a patient which may cause the airway to bend and form a kink, or may exert pressure upon the airway which can significantly or completely block the flow of air through the airway. This can reduce or eliminate the negative pressure being transmitted to the wound site which may cause an accumulation of excess wound exudate at the wound site and/or adversely affect the healing of the wound and, therefore, the wound outcome.

Further, many different types of wound dressings are known for aiding in negative pressure wound dressing systems. These different types of wound dressings include many different types of materials and layers, for example, gauze, pads, foam pads or multi-layer wound dressings. However, in each case, the dressing requires a suction port, typically in a backing layer of the dressing, to provide connection to an airway to transmit negative pressure from a pump to the wound site. The suction port is typically formed integral with the wound dressing. The location of the port therefore determines the location and orientation of the airway when the airway is attached to the port. Often, this means that the airway is orientated in a position which is disadvantageous for the patient, for example in a position which means that the airway is prone to being pressed against the skin of the patient or in a position where the patient is prone to obstructing the airway by resting upon the airway.

Further, the stiffness of the suction port in such close proximity to the wound site can adversely affect the healing process. Patient movement or pressure exerted onto the wound dressing may bring the healing wound into contact with the inflexible suction port of the dressing. Such force can cause disturbance of a wound bed which can damage a wound site. This can potentially cause delays in healing of the wound site and discomfort for the patient.

It is an object of embodiments of the present invention to at least partially overcome or alleviate the above problems and/or to provide an improved airway for a wound dressing.

### SUMMARY OF THE INVENTION

The present invention concerns an airway for a negative pressure wound dressing. The airway may comprise a strip of material having a first surface comprising at least one groove. The airway may comprise a cover, such as a film, arranged over the first surface of the strip of material. The cover, for example a film, may be adhered to the strip of material around its longitudinal edges. The at least one groove may be provided with at least one projection. The at least one projection may form at least two channels between the strip and the film.

According to a broad aspect of the present invention there is provided an airway for a negative pressure wound dressing, the airway comprising a strip of material having a first surface comprising at least one groove, and a film arranged over the first surface of the strip of material and adhered to the strip of material around its longitudinal edges to form at least one channel between the strip and the film.

Advantageously, the present invention provides an airway which benefits from the formation of at least one channel provided by the at least one groove in the strip which allows for the passage of air away from a wound site (for example when used as part of a negative pressure wound dressing). As such, the airway can be used to transmit a negative pressure to a wound site to facilitate wound healing.

In addition, the present invention benefits from the formation of the at least one channel by adhering the film to the strip of material. Comparatively, arrangements disclosed in the prior art typically require at least a spacer material and two film layers to form an airway. In the arrangements of the prior art, the two film layers must be carefully arranged over the spacer material, which itself must be carefully arranged centrally between the two film layers, so that adhering the longitudinal edges of the two films layers together does not adhere either film layer to the spacer material, which can adversely affect performance of the airway. The present invention, therefore, provides for an airway which is formed from fewer components than that of the prior art. Moreover, the present invention does not require a separate spacer material or similar to provide a channel for the passage of air, therefore, manufacture of the airway of the present invention is simpler than manufacture of airways of the prior art. Thus, the present invention is manufactured from fewer components and does not require the precise arrangement of components prior to adhering the films layers together as that of airways of the prior art. As such, manufacture of the present invention is easier and has fewer drawbacks than manufacture of airways of the prior art.

Moreover, in use, the airways of the prior art, in particular an airway formed using a spacer material, can be folded upon itself and compressed, for instance by movement of a user and, for example, the user lying on the airway. Due to the arrangement of the airway of the prior art, for example as described above, the airway of the prior art would be prone to forming a kink, compressing the two layers of waterproof material together which in turn compresses the spacer material. Disadvantageously this prevents, or at least significantly reduces, the flow of fluid (for example, air) along the airway, therefore, considerably reducing its ability to transmit negative pressure to a wound site. Advantageously, the present invention does not suffer from the same drawback. The provision of the at least one channel formed between the strip and the film means that even when the airway of the invention is folded and/or compressed by a user (or other means) the film does not compress into the at least one channel such that passage of fluid along the channel is prevented, or at least considerably reduced, and does not, therefore, prevent the airway of the present invention transmitting a negative pressure to a wound site.

Further advantageously, the airway of the invention prevents, or at least reduces the degree of, resonance in the airway when a fluid is passed along the airway.

The at least one groove may be provided with at least one projection to form at least two channels between the strip and the film.

Accordingly in a first aspect of the invention, there is provided an airway for a negative pressure wound dressing, the airway comprising a strip of material having a first surface comprising at least one groove, the airway further comprising a film arranged over the first surface of the strip of material and adhered to the strip of material around its longitudinal edges, wherein the at least one groove is provided with at least one projection to form at least two channels between the strip and the film.

The at least one groove may be provided with a plurality of projections, such that there is provided a plurality of channels between the strip and the film.

A plurality of channels has the advantage of increasing the number of routes available to fluid flowing along the airway. This means that, in the event the airway is bent, twisted or kinked, it is probable that flow of fluid along the airway would not be completely obstructed as at least some channels would remain at least partially open to the flow of fluid. Thus, the flow of fluid along the airway of the invention is maintained and is more reliable even if the airway is bent, twisted or kinked, compared to airways of the prior art.

A groove may be a space cut into the first surface of the strip of material.

The at least one projection may be formed integral with the strip of material. The at least one projection may be formed integral with the strip of material and the at least one projection may extend from a base of the or each groove.

The plurality of projections may comprise any number of projections, for example about 5, about 10, about 15, about 20, about 25, about 30, about 35, about 40, about 45, about 50, about 55, about 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, or about 250 projections.

The plurality of projections may comprise at least about 5, about 10, about 15, about 20, about 25, about 30, about 35, about 40, about 45, about 50, about 55, about 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, or at least about 250 projections.

The plurality of projections may comprise no more than about 5, about 10, about 15, about 20, about 25, about 30, about 35, about 40, about 45, about 50, about 55, about 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, or no more than about 250 projections.

At least some of the channels may be in fluid communication with one another.

Advantageously, this means that the overall channel volume of the airway and the number of routes that fluid can take when flowing along the airway is increased which in turn means that the flow of fluid to and/or from a wound site is increased. In some embodiments, all of the channels are in fluid communication with one another, which further enhances the aforementioned advantage.

The plurality of channels may form a network of interconnected channels.

The airway may comprise a first end comprising an orifice operable as an inlet or outlet for the flow of fluid. The airway may comprise a second end comprising an orifice operable as an inlet or outlet for the flow of fluid. The first end may comprise an orifice operable as an inlet or outlet for the flow of fluid, and the second end may comprise an orifice operable as the other of the inlet or outlet for the flow of fluid.

At least one, preferably all, of the channels may be in fluid communication with the orifice of the first end and/or the orifice of the second end.

The orifice of the first end may be in fluid communication with the orifice of the second end. The orifice of the first end may be in fluid communication with the orifice of the second end via the at least two channels. Advantageously, this allows the airway of the invention to comprise at least one appropriately configured connector at the first end and/or the second end.

The at least one groove may be a plurality of grooves.

The plurality of grooves may comprise any number of grooves, for example about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, about 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, or about 250 grooves.

The plurality of grooves may comprise at least about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, about 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, or at least about 250 grooves.

The plurality of grooves may comprise no more than about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, about 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, or no more than about 250 grooves.

In embodiments comprising a plurality of grooves, the passage of air along the airway away from the wound site may be more efficient than in embodiments comprising a single groove. That is not to say that the present invention when comprising a single groove is not advantageous compared to airways of the prior art.

The at least two channels may comprise any number of channels, for example about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, about 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, or about 250 channels.

The at least two channels may comprise at least about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, about 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, or at least about 250 channels.

The at least two channels may comprise no more than about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, about 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, or no more than about 250 channels.

In embodiments comprising a plurality of channels, the passage of air along the airway away from the wound site may be more efficient than in embodiments comprising a single channel. That is not to say that the present invention when comprising a single channel is not advantageous compared to airways of the prior art.

The at least one groove may be formed by heat stamping, laser cutting, laser engraving or thermo-forming. Beneficially, such methods of forming the at least one groove are reliable and cost effective.

In embodiments comprising a plurality of grooves, the plurality of grooves may be arranged as an interlaced pattern of grooves. By ‘interlaced’, we mean that each groove crosses at least one other groove. Beneficially, an interlaced pattern of grooves is less likely to be blocked by bending/folding compared to, for example, a spacer material or a plurality of grooves arranged in parallel to one another.

The interlaced pattern of grooves may comprise a plurality of criss-crossed grooves. By this we mean that there is significant crossing of each groove with at least one other groove. Advantageously, an interlaced pattern of grooves, for example comprising a plurality of criss-crossed grooves, increases the overall groove volume of the airway. Beneficially, this means that a greater volume of fluid (for example air) can be transmitted along the airway at any given time compared to airways of the prior art which do not comprise a plurality of grooves, in particular an interlaced pattern of grooves. Further advantageously, an interlaced pattern of grooves maintains a favourable flow rate of fluid along the airway during compression or load upon the airway. As such, for example when used for transmitting a negative pressure to a wound site, the airway of the invention maintains a negative pressure at a wound site even when the airway has been folded and/or compressed by a user.

The plurality of grooves may be arranged as a zig-zag pattern of grooves. By this we mean that each groove is arranged in a zig-zag orientation and that each groove does not cross another groove.

The strip of material may comprise a second surface which forms the opposite facing surface to that of the first surface. The second surface may not comprise any grooves. The second surface may be substantially planar.

The at least one projection may be formed of portions of the first surface of the strip of material which are not grooves.

The or each projection may extend from a base of the or each groove.

The or each projection may extend to a height equal to, or at least substantially equal to, the height of the strip of material.

The or each projection may abut the film when the film is adhered to the first surface of the strip of material. The film may be adhered to one or more of the at least one projections. For example, the or each projection may comprise an upper surface which is adhered to a lower surface of the film. The film may be adhered to one or more of the at least one projections by heat lamination. In known airways, despite comprising a spacer material, when in use, the films used to sandwich the spacer material often collapse inwardly toward each other causing an obstruction to the flow of fluid through the airway. Advantageously, in respect of the present invention, in use, the least one projection prevent the film collapsing into the at least one or plurality of grooves and therefore obstructing the flow of fluid through the airway.

The film may be substantially planar. Advantageously, this means that the airway has at least one substantially planar surface which makes the airway more comfortable should a user sit or rest upon the airway.

The at least one projections may be discrete, individual projections. The discrete, individual projections may each have a cross-section (when viewed in plan view) which is any shape but in particular may be circle, oval, triangle, square, diamond, rhombus, parallelogram, rectangle, pentagon, hexagon, heptagon or octagon.

The or each projection may be an elongated projection which extends at least part way along the length of the strip of material. The or each elongated projection may be a linear projection or may be in a zig-zag. The plurality of projections may comprise at least one discrete, separate projection and at least one elongated projection extending at least part way along the length of the strip of material.

The or each elongate projection may extend at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or at least about 95% along the length of the strip of material.

The strip of material may have a length of about 50 mm, about 60 mm, about 70 mm, about 80 mm, about 90 mm, about 100 mm, about 110 mm, about 120 mm, about 130 mm, about 140 mm, about 150 mm, about 160 mm, about 170 mm, about 180 mm, about 190 mm, about 200 mm, about 210 mm, about 220 mm, about 230 mm, about 240 mm, about 250 mm, about 260 mm, about 270 mm, about 280 mm, about 290 mm, about 300 mm, about 310 mm, about 320 mm, about 330 mm, about 340 mm, about 350 mm, about 360 mm, about 370 mm, about 380 mm, about 390 mm, or about 400 mm.

The strip of material may have a length of at least about 50 mm, about 60 mm, about 70 mm, about 80 mm, about 90 mm, about 100 mm, about 110 mm, about 120 mm, about 130 mm, about 140 mm, about 150 mm, about 160 mm, about 170 mm, about 180 mm, about 190 mm, about 200 mm, about 210 mm, about 220 mm, about 230 mm, about 240 mm, about 250 mm, about 260 mm, about 270 mm, about 280 mm, about 290 mm, about 300 mm, about 310 mm, about 320 mm, about 330 mm, about 340 mm, about 350 mm, about 360 mm, about 370 mm, about 380 mm, about 390 mm, or at least about 400 mm.

The strip of material may have a length of no more than about 50 mm, about 60 mm, about 70 mm, about 80 mm, about 90 mm, about 100 mm, about 110 mm, about 120 mm, about 130 mm, about 140 mm, about 150 mm, about 160 mm, about 170 mm, about 180 mm, about 190 mm, about 200 mm, about 210 mm, about 220 mm, about 230 mm, about 240 mm, about 250 mm, about 260 mm, about 270 mm, about 280 mm, about 290 mm, about 300 mm, about 310 mm, about 320 mm, about 330 mm, about 340 mm, about 350 mm, about 360 mm, about 370 mm, about 380 mm, about 390 mm, or no more than about 400 mm.

The strip of material may have a width of about 5 mm, about 10 mm, about 15 mm, about 20 mm, about 25 mm, about 30 mm, about 35 mm, about 40 mm, about 45 mm, or about 50 mm.

The strip of material may have a width of at least about 5 mm, about 10 mm, about 15 mm, about 20 mm, about 25 mm, about 30 mm, about 35 mm, about 40 mm, about 45 mm, or at least about 50 mm.

The strip of material may have a width of no more than about 5 mm, about 10 mm, about 15 mm, about 20 mm, about 25 mm, about 30 mm, about 35 mm, about 40 mm, about 45 mm, or no more than about 50 mm.

The strip of material may have a depth of about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, about 2.2 mm, about 2.3 mm, about 2.4 mm, about 2.5 mm, about 2.6 mm, about 2.7 mm, about 2.8 mm, about 2.9 mm or about 3.0 mm.

The strip of material may have a depth of at least about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, about 2.2 mm, about 2.3 mm, about 2.4 mm, about 2.5 mm, about 2.6 mm, about 2.7 mm, about 2.8 mm, about 2.9 mm or at least about 3.0 mm.

The strip of material may have a depth of no more than about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, about 2.2 mm, about 2.3 mm, about 2.4 mm, about 2.5 mm, about 2.6 mm, about 2.7 mm, about 2.8 mm, about 2.9 mm or no more than about 3.0 mm.

The depth of the strip of material is measured from the base of the material to its highest point, e.g., to the top of a projection.

The or each groove may have a depth of about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, about 2.2 mm, about 2.3 mm, about 2.4 mm, about 2.5 mm, about 2.6 mm, about 2.7 mm, about 2.8 mm, or about 2.9 mm.

The or each groove may have a depth of at least about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, about 2.2 mm, about 2.3 mm, about 2.4 mm, about 2.5 mm, about 2.6 mm, about 2.7 mm, about 2.8 mm, or at least about 2.9 mm.

The or each groove may have a depth of no more than about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, about 2.2 mm, about 2.3 mm, about 2.4 mm, about 2.5 mm, about 2.6 mm, about 2.7 mm, about 2.8 mm, or no more than about 2.9 mm.

The or each groove may have a depth of between about 0.1 mm and about 2.9 mm, about 0.2 mm and about 2.8 mm, about 0.3 mm and about 2.6 mm, about 0.4 mm and about 2.4 mm, about 0.5 mm and about 2.2 mm, about 0.5 mm and about 2.0 mm, about 0.6 mm and about 1.8 mm, about 0.7 mm and about 1.6 mm, about 0.8 mm and about 1.4 mm, about 0.9 mm and about 1.2 mm, or about 1.0 mm.

Advantageously, the or each groove has a depth which is significantly less than the depth of a spacer material commonly found in known airways. As such, the present invention provides for an airway which has reduced depth compared to that of airways of the prior art. Thus, the airway of the present invention is more comfortable for a patient should they lie upon the airway.

By ‘depth’ of a groove, we mean the distance between the uppermost surface or point of a projection and the base of the groove from which the projection extends.

The or each groove may have a width of about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, about 2.2 mm, about 2.3 mm, about 2.4 mm, about 2.5 mm, about 2.6 mm, about 2.7 mm, about 2.8 mm, about 2.9 mm or about 3.0 mm.

The or each groove may have a width of at least about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, about 2.2 mm, about 2.3 mm, about 2.4 mm, about 2.5 mm, about 2.6 mm, about 2.7 mm, about 2.8 mm, about 2.9 mm or at least about 3.0 mm.

The or each groove may have a width of no more than about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, about 2.2 mm, about 2.3 mm, about 2.4 mm, about 2.5 mm, about 2.6 mm, about 2.7 mm, about 2.8 mm, about 2.9 mm or no more than about 3.0 mm.

The or each groove may have a width of between about 0.1 mm and about 3.0 mm, about 0.2 mm and about 2.8 mm, about 0.3 mm and about 2.6 mm, about 0.4 mm and about 2.4 mm, about 0.5 mm and about 2.2 mm, about 0.5 mm and about 2.0 mm, about 0.6 mm and about 1.8 mm, about 0.7 mm and about 1.6 mm, about 0.8 mm and about 1.4 mm, about 0.9 mm and about 1.2 mm, or about 1.0 mm.

Advantageously, grooves having the above-mentioned depth and/or width dimensions allow for the sufficient flow of fluid (typically air) away from the wound site when used as part of a negative pressure wound dressing. Such dimensions of the grooves are typically smaller than the dimensions of a spacer material used in known airways. The present invention therefore provides an airway which may be less cumbersome for the patient and does not cause the patient discomfort should they lie upon the airway when in use.

Further advantageously, the abovementioned depth and/or width dimensions of the or each groove further reduce the ability of the film to compress into the or each groove if the airway is, for example, folded upon itself or is put under the weight of the user, when in use.

By ‘width’ of a groove, we mean the distance between adjacent projections, between which is a groove.

In embodiments comprising more than one groove, not all of the grooves may have the same width. The average width of the grooves may be about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, about 2.2 mm, about 2.3 mm, about 2.4 mm, about 2.5 mm, about 2.6 mm, about 2.7 mm, about 2.8 mm, about 2.9 mm or about 3.0 mm.

The average width of the grooves may be at least about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, about 2.2 mm, about 2.3 mm, about 2.4 mm, about 2.5 mm, about 2.6 mm, about 2.7 mm, about 2.8 mm, about 2.9 mm or at least about 3.0 mm.

The average width of the grooves may be no more than about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, about 2.2 mm, about 2.3 mm, about 2.4 mm, about 2.5 mm, about 2.6 mm, about 2.7 mm, about 2.8 mm, about 2.9 mm or no more than about 3.0 mm.

The average width of the grooves may be between about 0.1 mm and about 3.0 mm, about 0.2 mm and about 2.8 mm, about 0.3 mm and about 2.6 mm, about 0.4 mm and about 2.4 mm, about 0.5 mm and about 2.2 mm, about 0.5 mm and about 2.0 mm, about 0.6 mm and about 1.8 mm, about 0.7 mm and about 1.6 mm, about 0.8 mm and about 1.4 mm, about 0.9 mm and about 1.2 mm, or about 1.0 mm.

The grooves may form a grooved area. By ‘grooved area’ we mean the area of the strip of material generally encompassed by the grooves.

The grooves may form a grooved area that has a width which is equal to about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or substantially all, or all, of the width of the airway.

The grooves may form a grooved area that has a width which is equal to at least about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or at least substantially all, or all, of the width of the airway.

The grooves may form a grooved area that has a width which is no more than about 10%, about 15%, about 20%, about 25%, about 30%, about 35%, about 40%, about 45%, about 50%, about 55%, about 60%, about 65%, about 70%, about 75%, about 80%, about 85%, about 90% or no more than about 95% of the width of the airway.

The grooves may form a grooved area that has a length which is equal to about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or substantially all, or all, of the length of the airway.

The grooves may form a grooved area that has a length which is equal to at least about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or at least substantially all, or all, of the length of the airway.

The grooves may form a grooved area that has a length which is no more than about 10%, about 15%, about 20%, about 25%, about 30%, about 35%, about 40%, about 45%, about 50%, about 55%, about 60%, about 65%, about 70%, about 75%, about 80%, about 85%, about 90% or no more than about 95% of the length of the airway.

The film preferably has a thickness of at least about 0.05 mm, about 0.1 mm, about 0.15 mm, about 0.2 mm, about 0.25 mm, about 0.3 mm, about 0.35 mm, about 0.4 mm, about 0.45 mm, about 0.5 mm, about 0.55 mm, about 0.6 mm, about 0.65 mm, about 0.7 mm, about 0.75 mm, about 0.8 mm, about 0.85 mm, about 0.9 mm, about 0.95 mm, or at least about 1.0 mm.

The film preferably has a thickness of no more than about 0.05 mm, about 0.1 mm, about 0.15 mm, about 0.2 mm, about 0.25 mm, about 0.3 mm, about 0.35 mm, about 0.4 mm, about 0.45 mm, about 0.5 mm, about 0.55 mm, about 0.6 mm, about 0.65 mm, about 0.7 mm, about 0.75 mm, about 0.8 mm, about 0.85 mm, about 0.9 mm, about 0.95 mm, or no more than about 1.0 mm.

Each channel may be in fluid communication with at least one other channel. Each channel may be in fluid communication with each other channel such that the plurality of channels forms an interconnected lattice of channels in fluid communication with one another. Advantageously, this facilitates the passage of air (or another fluid) along the channels, therefore, providing an airway which reliably transmits a negative pressure to a wound site. Further advantageously, in the event that the airway is bent (i.e., kinked) in use, the arrangement of the channels maintains an effective flow of air (or another fluid) along the channels. Thus, obstruction of the airway is prevented, or at least significantly reduced compared to airways of the prior art.

The fluid may be any gas or liquid.

The plurality of channels may comprise at least a first channel and a second channel.

The first channel and the second channel may be distinct from one another such that the first channel is not in fluid communication with the second channel. This arrangement provides an airway which allows for the passage of fluid in two directions, i.e., the passage of fluid away from a wound site and the passage of fluid toward a wound site.

The plurality of channels may comprise at least a first channelled area and a second channelled area, each channelled area comprising two or more channels. The first channelled area may be distinct from the second channelled area such that the first channelled area is not in fluid communication with the second channelled area.

The first channelled area and the second channelled area may be separated by a seal between the strip and the film. This arrangement provides an airway which allows for the passage of fluid in two directions, i.e., the passage of fluid away from a wound site and the passage of fluid toward a wound site. The seal may be an adhesion between the film and one or more projections. The seal may penetrate into one or more grooves. The seal may be formed by lamination of the film to the strip. The seal may be a longitudinal seal extending lengthways along the airway and arranged to separate the first channelled area from the second channelled area so that the two channelled areas are not in fluid communication with one another. In this embodiment, the airway may comprise two tubular portions at an end, for example the first end, and a first tubular portion may be arranged in fluid communication with the first channelled area, and a second tubular portion may be arranged in fluid communication with the second channelled area. Advantageously, this means that the airway can provide the passage of fluid in two different directions, i.e., a first direction, for example toward the wound site, via the first channelled area, and a second direction, for example away from the wound site, via the second channelled area.

The film and the strip may be formed of the same material. Beneficially, this means that the airway can be formed from cost effective materials that are readily obtainable and tends to improve the adhesion of one material to the other.

The film may be formed of a material selected from the group comprising polyurethane, thermoplastic polyurethane, thermoplastic elastomer, polyethylene, polypropylene, polyvinyl chloride, ethylene-vinyl acetate and a silicone material, for example a silicone rubber.

The strip may be formed of a material selected from the group comprising polyurethane, thermoplastic polyurethane, thermoplastic elastomer, polyethylene, polypropylene, polyvinyl chloride, ethylene-vinyl acetate and a silicone material, for example a silicone rubber.

Advantageously, each of these materials are cheap and easily obtainable. Further advantageously, each of these materials provide for an airway which is able to be stretched, is flexible and which is relatively soft. As such, the airway does not irritate a patient if the patient's movement causes the airway to repeatedly rub on their skin. Moreover, these materials, in particular polyurethane, are reusable and recyclable.

The film may be adhered to the strip of material by lamination. Lamination of the film to the strip may be by heat lamination, pressure lamination, heat and pressure lamination, welding or an adhesive.

The film and the strip may each be formed of a recyclable material. Beneficially, using a recyclable material to form the airway is environmentally friendly.

The airway may have a generally planer exterior surface.

The strip of material and/or the film may be formed of a transparent or translucent material. Advantageously, this means that the airway is transparent or translucent, respectively, so that if a blockage occurs within the airway, for example clogging due to wound exudate, then this is visible by the patient or a healthcare professional.

The strip of material may have any shape but may preferably be the shape of an elongated rectangle comprising a first longitudinal edge arranged parallel to a second longitudinal edge. The longitudinal edges may be joined by two curved opposing ends. At least one of the two curved ends may be tapered.

The airway may comprise a first end. The first end may comprise a filter, an infection detection indicator and/or a dressing change indicator.

The filter may be impermeable to liquids but permeable to gases. Advantageously, the filter may function as a liquid barrier to ensure that no wound exudate can exit the wound dressing.

The filter may function as a bacterial barrier.

The filter may be a microporous membrane. The microporous membrane may be any polymeric material. The filter may be a microporous hydrophobic membrane (MHM). The MHM may be formed from one or more of PTFE, polypropylene, PVDF and acrylic copolymer. Each of these optional polymers can be treated in order to obtain specific surface characteristics that can be both hydrophobic and oleophobic. As such, these will repel liquids with low surface tensions such as multi-vitamin infusions, lipids, surfactants, oils and organic solvents.

The filter may comprise an odour absorbent material, for example activated charcoal or carbon fibre cloth.

The filter may have a pore size of about 0.1 μm, about 0.2 μm, about 0.3 μm, about 0.4 μm, about 0.5 μm, about 0.6 μm, about 0.7 μm, about 0.8 μm, about 0.9 μm or about 1.0 μm.

The filter may comprise an oleophobic filter membrane. Beneficially, the oleophobic filter membrane may prevent any lipids found in wound exudate from blocking the filter.

The infection detection indicator and/or the dressing change indicator may comprise an auxiliary compound. The auxiliary compound may be an antimicrobial, antifungal, anti-inflammatory, or any other such therapeutic compounds. In some embodiments, the indicator may be by means of a colour change. The colour may change as a function of time (e.g., to indicate when the dressing needs to be changed), if the dressing is saturated, or if the dressing has absorbed a certain amount of a harmful substance (e.g., to indicate the presence of infectious agents).

The first end may comprise a viewing window through which the infection detection indicator and/or the dressing change indicator can be viewed. The viewing window may be monitored electronically and may be used in conjunction with a computer program or system to alert a patient or physician to the saturation level of the dressing.

An end, for example the first end, of the airway may comprise a tapered portion extending away from the airway.

The tapered portion may comprise a tubular portion. The tubular portion may extend away from the first end to a greater extent than that of the tapered portion. The tubular portion may be configured to receive a tube or Luer or barb connector which may be connectable to a pump. The tubular portion may have a depth greater than that of any other portion of the airway. As such, the tubular portion may be more rigid than any other portion of the airway. Advantageously, this facilities insertion of a tube or Luer or barb connector. Further advantageously, the tubular portion, in particular in embodiments comprising a tubular portion which is more rigid than any other portion of the airway, prevents the airway kinking at the end comprising the tubular portion, for example at the first end. The tubular portion may extend for no more than a length equal to about 2%, about 4%, about 6%, about 8% or about 10% of the length of the airway.

In embodiments of the invention which do not comprise a tubular portion, a tube or Luer or barb connector may be adhered (for example by using an adhesive) to the first surface of the strip of material at an end of the strip (for example, the first end) prior to adhering the film to the first surface of the strip of material. Thus, the tube or Luer or barb connector is sandwiched between the first surface of the strip of material and the film.

The first end may comprise a connector for connecting the airway to a pump. The connector may connect the airway to the pump via medical tubing. In embodiments where the airway is used as part of a negative pressure wound dressing, the connector may be configured to transmit negative pressure from a pump to the wound dressing for the application of topical negative pressure at a wound site.

The connector may comprise a body having an upper surface and a lower surface, each surface being joined together at their periphery. The connector may comprise a nozzle having a mouth at its end. The nozzle may extend away from the body of the connector at the join of the upper surface to the lower surface. The nozzle may be tapered to facilitate connection to a tube or other connector or adapter, which may in turn be connected to a pump.

The connector may comprise an orifice. The lower surface of the connector may comprise the orifice. The orifice may be arranged centrally relative to the periphery of the lower surface of the connector. The orifice may be in fluid communication with the nozzle and the mouth.

In embodiments of the invention which do not comprise a tubular portion, the airway may comprise one or more apertures, for example in the shape of a cross or circle, preferably at a first end of the airway and preferably in a second surface of the strip of material so that the film does not comprise the one or more apertures. Thus, the one or more apertures are arranged in fluid communication with the at least one channel. In use, the connector may be adhered to the airway, for example by using an adhesive or by heat welding, at the second surface of the strip of material such that the orifice is in fluid communication with the one or more apertures. As such, the mouth, nozzle, orifice, the one or more apertures and the at least one channel are in fluid communication with one another. A Luer or barb connector may be connected to the nozzle of the connector and connected to a pump. Activation of the pump, therefore, allows for the passage of air from a wound site, along the at least one channel, through the one or more apertures, through the orifice, nozzle and mouth, and through the Luer or barb connector toward the pump. As such, a negative pressure is generated at the wound site.

The filter, the infection detection indicator and/or the dressing change indicator may be formed integral with the connector. The filter, the infection detection indicator and/or the dressing change indicator may be positioned within the nozzle of the connector.

The filter, the infection detection indicator, the dressing change indicator and/or connector may be attached to the first surface of the strip of material.

The filter, the infection detection indicator, the dressing change indicator and/or connector may be attached to a second surface of the strip of material.

The filter, the infection detection indicator, the dressing change indicator and/or connector may be attached to the second surface of the strip of material and in fluid connection with the first surface via the one or more apertures.

The airway may comprise a second end distal to the first end. The second end may be substantially the same size and shape as the first end, or may be enlarged relative to the size of the first end. The second end may comprise a lip. Advantageously, the lip increases the surface area of the second end for adhering an adhesive. The second end may be any shape, for example circular, triangular, square, pentagonal. Beneficially, an enlarged second end provides an adequate surface for an adhesive panel to be adhered to the airway.

The second end may comprise an adhesive panel for adhering the airway to a wound dressing. The adhesive panel may be a double-sided adhesive panel. The adhesive panel may be separate to the airway until it is required to be adhered to the second end of the airway. Advantageously, the adhesive panel is suitable for adhering the airway to a wound dressing, for example a backing layer.

The strip of material may be formed by compression moulding or injection moulding. Each moulding technique provides a cost-effective method of forming the airway. Furthermore, compression moulding and injection moulding allow custom shapes and designs of the strip of material to be produced. Beneficially, this means that the number, shape and configuration of the grooves and any corresponding projections can be easily formed when forming the strip of material.

The strip of material may be a first strip of material.

The airway may comprise a second strip of material. The second strip of material may comprise any of the features, optional or otherwise, associated with the (first) strip of material as described above.

The airway may comprise a second strip of material having a first surface comprising at least one groove but preferably a plurality of grooves. The first surface of the second strip of material may be arranged over the film and adhered to the film around its longitudinal edges to form at least one, but preferably a plurality, of channels between the second strip of material and the film. Advantageously, this arrangement allows for at least two channels (i.e., at least a first channel and at least a second channel) to be formed which are not in fluid communication with one another. This arrangement provides an airway which allows for the passage of fluid in two directions, i.e., the passage of fluid away from a wound site and the passage of fluid toward a wound site.

Thus, in some embodiments, there may be provided an airway for a negative pressure wound dressing, the airway comprising a first strip of material having a first surface comprising at least one groove, the airway further comprising a film arranged over the first surface of the first strip of material and adhered to the first strip of material around its longitudinal edges, wherein the at least one groove is provided with at least one projection to form at least two channels between the first strip and the film, wherein the airway further comprises a second strip of material having a first surface comprising at least one groove provided with at least one projection, the first surface of the second strip of material being arranged over the film and adhered to the film around its longitudinal edges to form at least two channels, between the second strip and the film.

In some embodiments, there may be provided an airway for a negative pressure wound dressing, the airway comprising a first strip of material having a first surface comprising at least one groove, the airway further comprising a film arranged over the first surface of the first strip of material and adhered to the first strip of material around its longitudinal edges, wherein the at least one groove is provided with at least one projection to form at least two channels between the first strip and the film, wherein the airway further comprises a second strip of material having a first surface comprising at least one groove provided with at least one projection, the first surface of the second strip of material being arranged over the film and adhered to the film around its longitudinal edges to form at least two channels, between the second strip and the film, wherein the at least one groove comprised in the first strip of material is formed by heat stamping, laser cutting, laser engraving or thermo-forming.

In some embodiments, there may be provided an airway for a negative pressure wound dressing, the airway comprising a first strip of material having a first surface comprising at least one groove, the airway further comprising a film arranged over the first surface of the first strip of material and adhered to the first strip of material around its longitudinal edges, wherein the at least one groove is provided with at least one projection to form at least two channels between the first strip and the film, wherein the airway further comprises a second strip of material having a first surface comprising at least one groove provided with at least one projection, the first surface of the second strip of material being arranged over the film and adhered to the film around its longitudinal edges to form at least two channels, between the second strip and the film, wherein the at least one groove comprised in the second strip of material is formed by heat stamping, laser cutting, laser engraving or thermo-forming.

In some embodiments, there may be provided an airway for a negative pressure wound dressing, the airway comprising a first strip of material having a first surface comprising at least one groove, the airway further comprising a film arranged over the first surface of the first strip of material and adhered to the first strip of material around its longitudinal edges, wherein the at least one groove is provided with at least one projection to form at least two channels between the first strip and the film, wherein the airway further comprises a second strip of material having a first surface comprising at least one groove provided with at least one projection, the first surface of the second strip of material being arranged over the film and adhered to the film around its longitudinal edges to form at least two channels, between the second strip and the film, wherein the at least one groove comprised in the first strip of material is formed by heat stamping, laser cutting, laser engraving or thermo-forming, and the at least one groove comprised in the second strip of material is formed by heat stamping, laser cutting, laser engraving or thermo-forming.

The second strip of material may be formed of the same material as the first strip of material.

The airway may comprise at least one seal arranged to separate the plurality of channels into two or more distinct, separate channelled areas, for example a first channelled area and a second channelled area. In this embodiment, the at least one seal may be formed after the film has been adhered to the strip of material. Each separate channelled area may not be in fluid communication with another distinct, separate channelled area. The airway may comprise one, two, three or more than three seals. The seal may be formed between the film and the strip of material. The seal may be formed by heat lamination, pressure lamination, heat and pressure lamination, welding or an adhesive. Each separate channelled area may comprise at least one channel. Advantageously, in this embodiment, the airway may be used to provide multi-directional flow of fluid. For example, fluid can flow toward a wound site via the at least one channel in the first channelled area, and fluid can simultaneously flow away from a wound site via the at least one channel in the second channelled area.

The airway may be used with a wound dressing for the application of negative pressure therapy, positive pressure therapy, oxygen therapy or deep wound filling therapy, for example. The airway may also be used as part of a drug delivery system or infusion system.

As an example of an embodiment of the present invention in use, a typical negative pressure wound dressing comprises a pump for generating negative pressure, a dressing for covering and protecting a wound, the dressing comprising a wound contact layer, a backing layer and at least one layer of absorbent material between the wound contact layer and the backing layer. In use, a fluid communication pathway is provided from the wound, through the wound contact layer and through one or more layers of absorbent material disposed in the dressing. The fluid communication pathway may also extend through an opening in the backing layer. Advantageously, the fluid communication pathway can be formed at any suitable location on the wound dressing.

The airway may be adhered, for example via a double-sided adhesive at a second end of the airway, to the wound dressing between the backing layer and the at least one layer of absorbent material. Alternatively, provided the fluid communication pathway extends through an opening in the backing layer, the airway may be adhered to the wound dressing on the backing layer and an aperture in the strip of material of the airway and an aperture in the backing layer arranged concentrically.

A Luer or barb connector is inserted into a tubular portion of the first end of the strip of material, and the connector is connected to a pump, possibly via a tube or similar. Activation of the pump draws air from the wound site, through the wound contact layer and absorbent material via the fluid communication pathway, through the aperture of the adhesive panel, through the aperture at the second end of the strip of material, along the plurality of channels, through the tubular portion and Luer connector, and toward the pump. Thus, a negative pressure is created at the wound site.

The second strip of material may be an elongated strip of material. The second strip of material may comprise a first end. The second strip of material may comprise a second end. The second elongated strip of material may comprise a pair of longitudinal edges, i.e., a first longitudinal edge and a second longitudinal edge. The longitudinal edges may be joined at the first end by an outwardly curved portion, the outermost part of which may form the terminus of the first end. The longitudinal edges may be joined by an outwardly curved portion, the outermost part of which may form the terminus of the second end. The second end may be distal to the first end.

The second strip of material may have a width (i.e., the distance between the first longitudinal edge and the second longitudinal edge) of about 15 mm and a length (i.e., the distance from the terminus of the first end to the terminus of the second end) of about 300 mm.

In embodiments comprising a second strip of material, the first strip of material may comprise a first orifice at the first end, the first orifice being operable as an inlet or outlet for the flow of fluid. In embodiments comprising a second strip of material, the first strip of material may comprise a second orifice at the second end, the second orifice being operable as an inlet or outlet for the flow of fluid. The first orifice may be operable as an inlet or outlet for the flow of fluid, and the second orifice may be operable as the other of the inlet or outlet for the flow of fluid.

The first orifice may be in fluid communication with the second orifice. Advantageously, this allows the airway of the invention to comprise at least one appropriately configured connector at the first end and/or the second end.

The airway may comprise a third orifice. The second strip of material may comprise at least one orifice. The third orifice may be arranged at the first end of the second strip of material. The third orifice may be in the shape of a cross. The third orifice may be arranged central relative to the periphery of the first end of the second strip of material. The third orifice may extend through the entire of the depth of the second strip of material.

The airway may comprise a fourth orifice. The fourth orifice may be arranged at the second end of the second strip of material. The fourth orifice may be in the shape of a cross. The fourth orifice may be arranged central relative to the periphery of the second end of the second strip of material. The fourth orifice may extend through the entire of the depth of the second strip of material.

The second strip of material may comprise a downwardly facing first surface and an upwardly facing second surface, when in use. In use, the first surface may face toward the wound site and the second surface may face away from the wound site.

The first surface of the second strip of material may comprise a grooved area comprising at least one groove, preferably a plurality of grooves. The grooved area may comprise at least one projection, preferably a plurality of projections.

The grooved area of the first surface of the second strip of material may comprise each of the features of the grooved area of the first surface of the first strip of material.

The grooved area may have a width which is equal to about 60% of the width of the second strip of material, and a length which is equal to about 90% of the length of the second strip of material. The grooved area may be substantially rectangular.

The at least one groove may be arranged as an interlaced pattern of criss-crossed grooves, with each groove being separated from another groove, or another portion of a groove, by a projection. The or each projection may extend from the base of a groove. The or each groove may have a width of 1.0 mm and a depth of 1.5 mm. The or each groove may be formed by laser engraving/etching of the second strip of material. The second strip of material may have a depth of 2.0 mm.

In embodiments comprising a plurality of grooves, each groove may be in fluid communication with each other groove such that the plurality of grooves forms an interconnected lattice of grooves in fluid communication with one another. Advantageously, in this embodiment, the plurality of grooves increases the overall volume of the grooves of the second strip of material of the airway. In turn, this means that a greater volume of fluid (for example air) is transmitted along the second strip of material of the airway at any given time compared to airways of the prior art which do not comprise a plurality of grooves, in particular an interlaced pattern of grooves.

Further, when in use, the interconnected lattice of grooves maintains a favourable flow rate of fluid along the second strip of material of the airway during compression or load upon the airway. As such, for example when used for transmitting a negative pressure to a wound site, the airway of the invention maintains a negative pressure at a wound site even when the airway has been folded and/or compressed by a user.

The at least one groove may be in fluid communication with the third orifice. The at least one groove may be in fluid communication with the fourth orifice.

The airway may comprise the second strip of material such that first surface of the second strip of material is arranged over the film. The first surface of the second strip of material may be adhered to the film (for example by an adhesive, alternatively heat lamination may be used) about its longitudinal edges. The plurality of grooves may form at least one second channel, preferably a second plurality of channels, between the second strip of material and the film. In embodiments comprising a second plurality of channels, the second plurality of channels may be in fluid communication with each other channel such that the second plurality of channels forms a network of interconnected channels in fluid communication with one another. The interconnected channels may be in fluid communication with the third orifice at the first end of the second strip of material and/or with the fourth orifice at the second end of the second strip of material.

Advantageously, this arrangement allows for at least two channels (i.e., at least a first channel and at least a second channel) to be formed which are not in fluid communication with one another. This arrangement provides an airway which allows for the passage of fluid in two directions, i.e., the passage of fluid away from a wound site and the passage of fluid toward a wound site.

The at least one groove comprised in the first surface of the first strip of material may be provided with a plurality of projections, such that there is provided a plurality of channels between the first strip and the film.

The at least one groove comprised in the first surface of the second strip of material may be provided with a plurality of projections, such that there is provided a plurality of channels between the second strip and the film.

The at least one groove comprised in the first surface of the first strip of material may be provided with a plurality of projections, such that there is provided a plurality of channels between the first strip and the film, and the at least one groove comprised in the first surface of the second strip of material may be provided with a plurality of projections, such that there is provided a plurality of channels between the second strip and the film.

Thus, in some embodiments, there may be provided an airway for a negative pressure wound dressing, the airway comprising a first strip of material having a first surface comprising at least one groove, the airway further comprising a film arranged over the first surface of the first strip of material and adhered to the first strip of material around its longitudinal edges, wherein the at least one groove is provided with at least one projection to form at least two channels between the first strip and the film, wherein the airway further comprises a second strip of material having a first surface comprising at least one groove provided with at least one projection, the first surface of the second strip of material being arranged over the film and adhered to the film around its longitudinal edges to form at least two channels, between the second strip and the film, wherein the at least one groove comprised in the first surface of the first strip of material is provided with a plurality of projections, such that there is provided a plurality of channels between the first strip and the film, and/or wherein the at least one groove comprised in the first surface of the second strip of material is provided with a plurality of projections, such that there is provided a plurality of channels between the second strip and the film.

In some embodiments, there may be provided an airway for a negative pressure wound dressing, the airway comprising a first strip of material having a first surface comprising at least one groove, the airway further comprising a film arranged over the first surface of the first strip of material and adhered to the first strip of material around its longitudinal edges, wherein the at least one groove is provided with at least one projection to form at least two channels between the first strip and the film, wherein the airway further comprises a second strip of material having a first surface comprising at least one groove provided with at least one projection, the first surface of the second strip of material being arranged over the film and adhered to the film around its longitudinal edges to form at least two channels, between the second strip and the film, wherein the at least one groove comprised in the first surface of the first strip of material is provided with a plurality of projections, such that there is provided a plurality of channels between the first strip and the film, and/or wherein the at least one groove comprised in the first surface of the second strip of material is provided with a plurality of projections, such that there is provided a plurality of channels between the second strip and the film, wherein at least two channels between the first strip of material and the film are in fluid communication with one another, and/or wherein at least two channels between the second strip of material and the film are in fluid communication with one another.

In some embodiments, there may be provided an airway for a negative pressure wound dressing, the airway comprising a first strip of material having a first surface comprising at least one groove, the airway further comprising a film arranged over the first surface of the first strip of material and adhered to the first strip of material around its longitudinal edges, wherein the at least one groove is provided with at least one projection to form at least two channels between the first strip and the film, wherein the airway further comprises a second strip of material having a first surface comprising at least one groove provided with at least one projection, the first surface of the second strip of material being arranged over the film and adhered to the film around its longitudinal edges to form at least two channels, between the second strip and the film, wherein the at least one groove comprised in the first surface of the first strip of material is provided with a plurality of projections, such that there is provided a plurality of channels between the first strip and the film, and/or wherein the at least one groove comprised in the first surface of the second strip of material is provided with a plurality of projections, such that there is provided a plurality of channels between the second strip and the film, wherein at least two channels between the first strip of material and the film are in fluid communication with one another, and/or wherein at least two channels between the second strip of material and the film are in fluid communication with one another, wherein the channels between the first strip of material and the film form a network of interconnected channels, and/or wherein the channels between the second strip of material and the film form a network of interconnected channels.

In some embodiments, the channel(s) between the first strip of material and the film are not in fluid communication with the channel(s) between the second strip of material and the film.

Advantageously, the embodiment provides an airway which allows for the passage of fluid, for example air, in two directions simultaneously, i.e., the passage of fluid away from a wound site and the passage of fluid toward a wound site. Thus, this may facilitate wound healing through a number of mechanisms, including removal of excess exudate, reduction in periwound edema and increased perfusion, therefore, resulting in improved wound outcomes.

The airway may comprise at least one connector. The at least one connector may comprise a first connector for connection of the channel(s) between the first strip of material and the film to a first medical tubing and an interior lumen therebetween. The first connector may be connected, for example by an adhesive or heat welding, to the first end of the first strip of material such that the connector is in fluid communication with the first orifice.

The airway may comprise a second connector for connection of the channel(s) between the second strip of material and the film to a second medical tubing and an interior lumen therebetween. The second connector may be connected, for example by an adhesive or heat welding, to the first end of the second strip of material such that the connector is in fluid communication with the third orifice.

The first and/or second connector may be a Luer or barb connector. The first and/or second connector may be connectable to a pump.

The first orifice of the first strip of material and the second orifice of the first strip of material may be in fluid communication, via the or each first channel.

The third orifice of the second strip of material and the fourth orifice of the second strip of material may be in fluid communication, via the or each second channel.

The first and/or second orifice may not be in fluid communication with the third and/or fourth orifice.

The first orifice may be separated from the third orifice by the film. The first orifice may be separated from the third orifice by the film such that the first orifice is not in fluid communication with the third orifice.

The second orifice may be separated from the fourth orifice by the film. The second orifice may be separated from the fourth orifice by the film such that the second orifice is not in fluid communication with the fourth orifice.

The film between the first strip of material and the second strip of material may comprise a plurality of layers of film bonded together, for example by heat lamination or an adhesive.

In some embodiments, the film is formed of a material selected from the group comprising polyurethane, thermoplastic polyurethane, thermoplastic elastomer, polyethylene, polypropylene, polyvinyl chloride, ethylene-vinyl acetate and a silicone material.

The or each first orifice, second orifice, third orifice and/or fourth orifice may comprise a plurality of orifices.

The first end of the first and/or second strip of material may comprise from 2 to 18 orifices, from 6 to 15 orifices, or from 9 to 12 orifices.

The first end of the first and/or second strip of material may comprise about 2, about 3, about 4, about 5, about 6, about 7, about 8, about 9, about 10, about 11, about 12, about 13, about 14, about 15, about 16, about 17 or about 18 orifices.

The first end of the first and/or second strip of material may comprise at least about 2, about 3, about 4, about 5, about 6, about 7, about 8, about 9, about 10, about 11, about 12, about 13, about 14, about 15, about 16, about 17 or at least about 18 orifices.

The first end of the first and/or second strip of material may comprise no more than about 2, about 3, about 4, about 5, about 6, about 7, about 8, about 9, about 10, about 11, about 12, about 13, about 14, about 15, about 16, about 17 or no more than about 18 orifices.

The shape of the or each orifice may be a circle, square, rectangle, rhombus, triangle, pentagon, hexagon, heptagon, octagon, or in the shape of a cross or in the form of a slit.

The or each orifice may have a diameter or a longest dimension of about 0.1 cm, about 0.2 cm, about 0.3 cm, about 0.4 cm, about 0.5 cm, about 0.6 cm, about 0.7 cm, about 0.8 cm, about 0.9 cm, or about 1.0 cm.

The or each orifice may have a diameter or a longest dimension of at least about 0.1 cm, about 0.2 cm, about 0.3 cm, about 0.4 cm, about 0.5 cm, about 0.6 cm, about 0.7 cm, about 0.8 cm, about 0.9 cm, or at least about 1.0 cm.

The or each orifice may have a diameter or a longest dimension of no more than about 0.1 cm, about 0.2 cm, about 0.3 cm, about 0.4 cm, about 0.5 cm, about 0.6 cm, about 0.7 cm, about 0.8 cm, about 0.9 cm, or no more than about 1.0 cm.

According to a second aspect of the invention, there is provide the use of an airway according to the first aspect with a negative pressure wound dressing.

According to a third aspect of the invention, there is provided a negative pressure wound dressing comprising the airway according to the first aspect.

According to a fourth aspect of the invention, there is provided a method of manufacturing an airway comprising the steps:
a. Providing a strip of material having a first surface comprising at least one groove;
b. Arranging a film over the first surface of the strip of material; and
c. Adhering the film to the first surface of the strip of material around its longitudinal edges to form a plurality of channels between the strip and the film.

Prior to step (a), the at least one groove may be formed in the strip of material by heat stamping, laser cutting, laser engraving/etching or thermo-forming.

The strip of material may be formed by compression moulding or injection moulding.

The at least one groove may be a plurality of grooves.

In step (c), the film may be adhered to the strip of material by lamination. Lamination of the film to the strip may be by heat lamination, pressure lamination, heat and pressure lamination, welding or an adhesive.

According to a fifth aspect of the invention, there is provided a method of using a wound dressing comprising the airway according to the first aspect.

The method may comprise adhering the wound dressing to a wound site, preferably to the periwound skin.

The wound dressing may be a negative pressure wound dressing.

The invention according to any of the first to fifth aspects may of course individually include any one or more of the features, optional or otherwise, of one another.

### DETAILED DESCRIPTION OF THE INVENTION

In order that the invention may be more clearly understood one or more embodiments thereof will now be described, by way of example only, with reference to the accompanying drawings, of which:

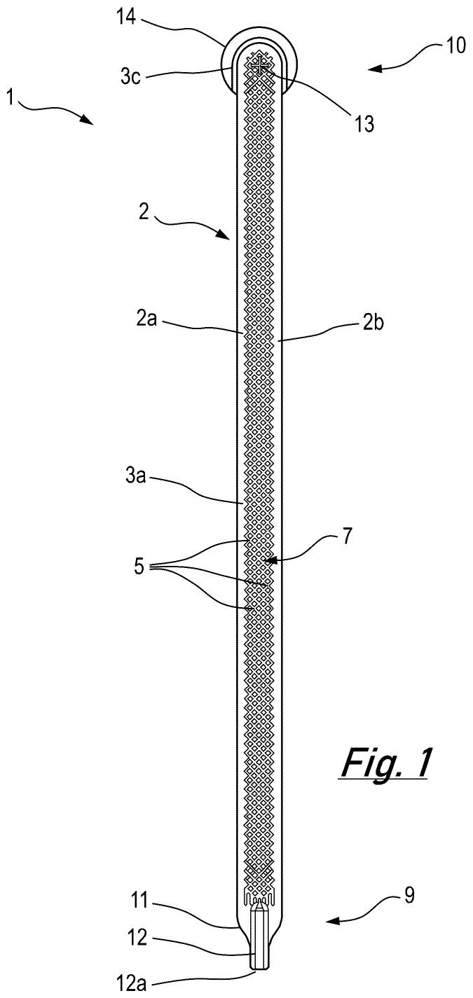

FIG. 1 is a plan view of a first embodiment of an airway;

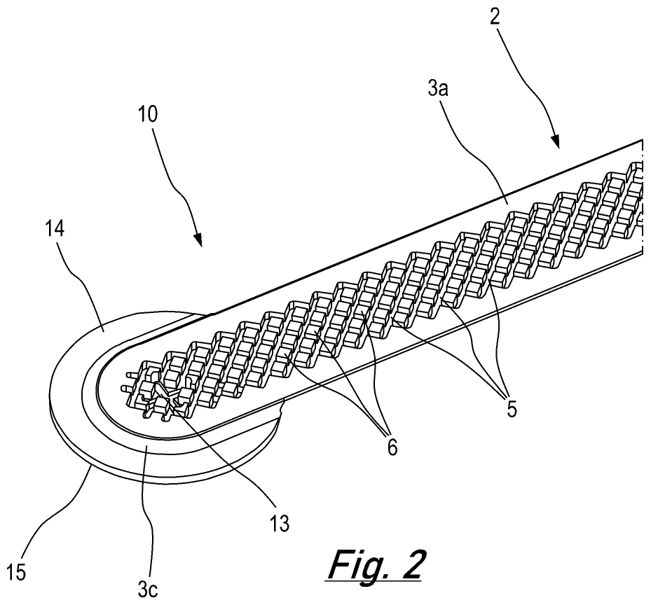

FIG. 2 is a perspective view of a portion of the airway of FIG. 1;

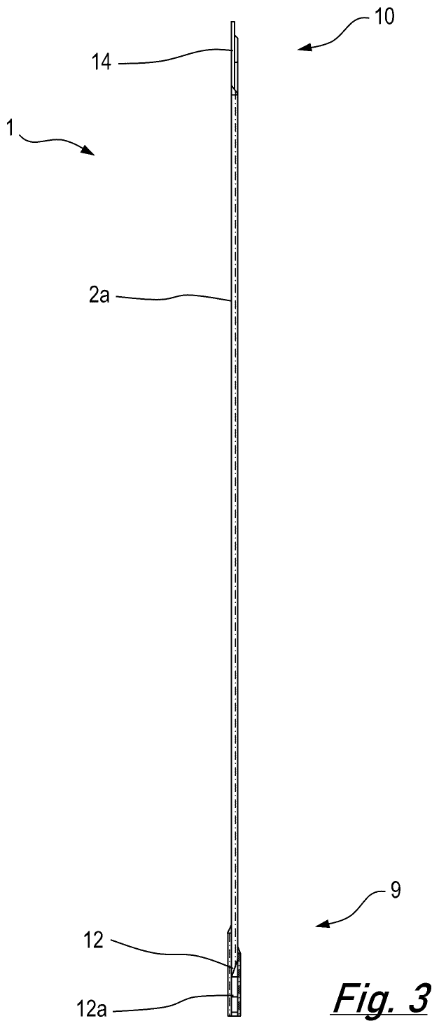

FIG. 3 is a side view of the airway of FIG. 1;

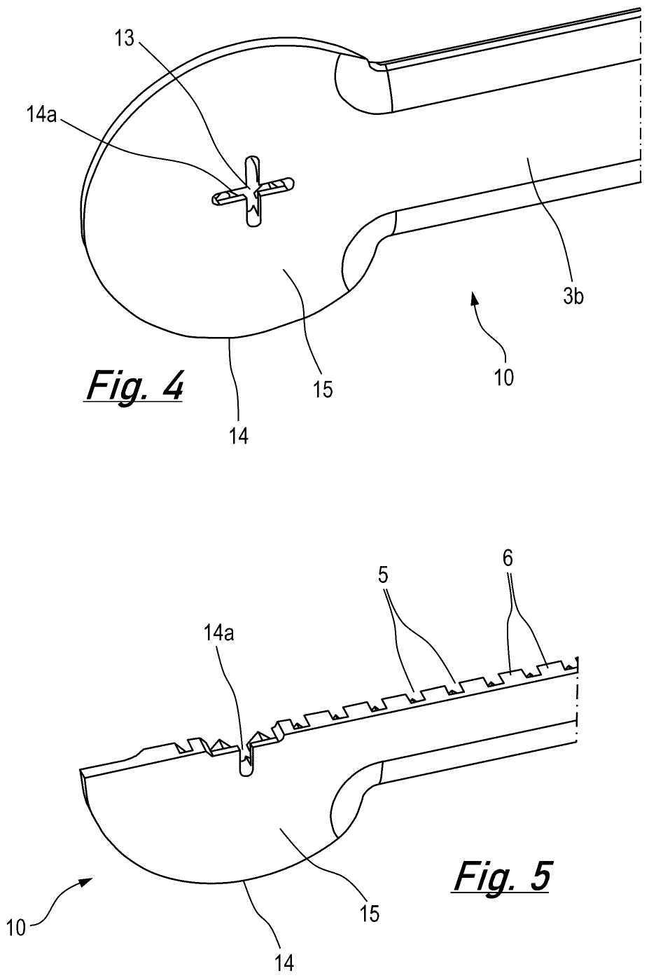

FIG. 4 is a reverse perspective view of the portion of the airway of FIG. 2;

FIG. 5 is a cut-away view of the airway shown in FIG. 4;

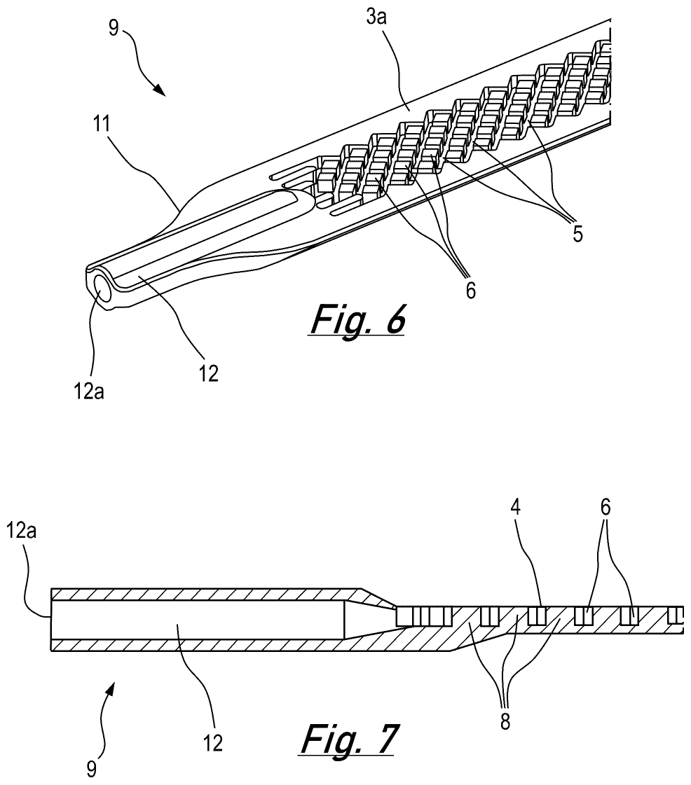

FIG. 6 is a perspective view of a portion of the airway of FIG. 1;

FIG. 7 is a side view of the portion of the airway of FIG. 6 where a film is adhered to the first surface of the strip of material;

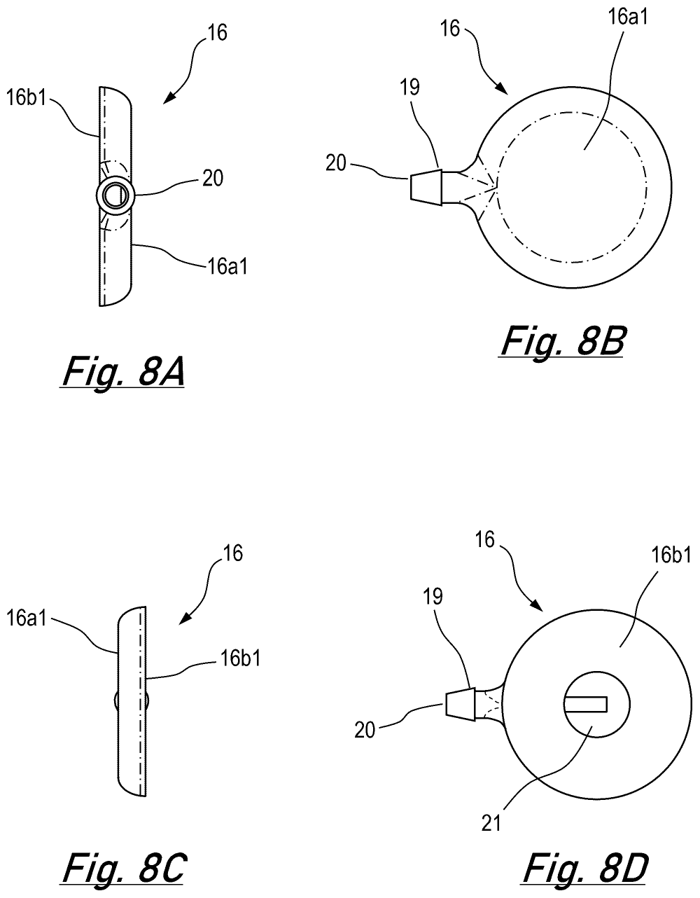

FIG. 8A is a front view of an example of a connector to be used with an airway;

FIG. 8B is a plan view of the connector of FIG. 8A;

FIG. 8C is a rear view of the connector of FIG. 8A;

FIG. 8D is a bottom view of the connector of FIG. 8A;

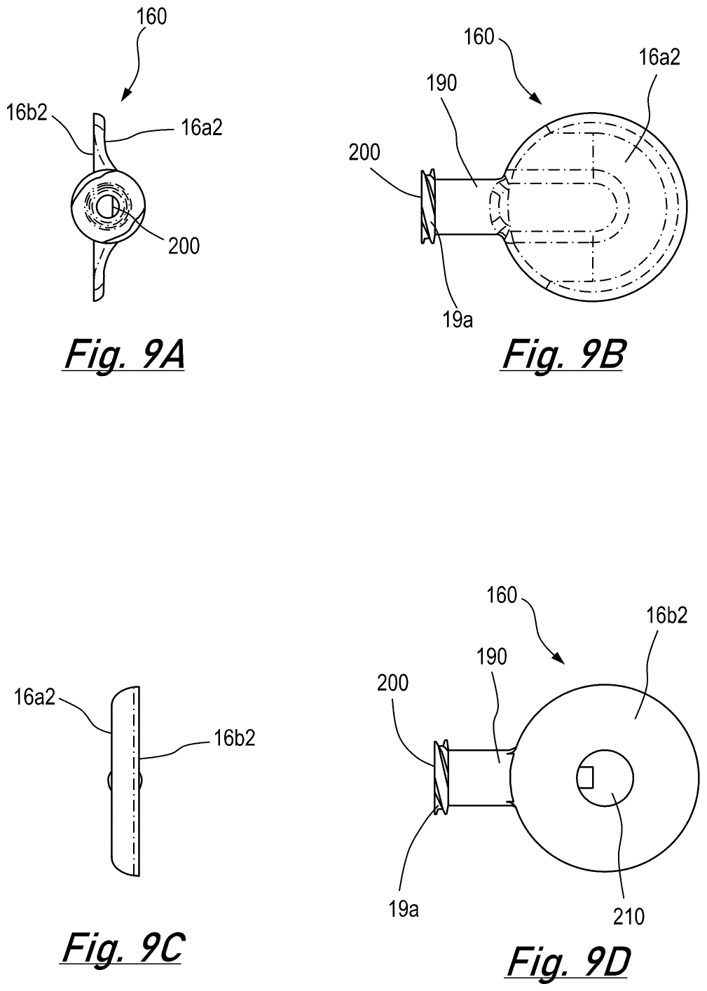

FIG. 9A is a front view of a further example of a connector to be used with an airway;

FIG. 9B is a plan view of the connector of FIG. 9A;

FIG. 9C is a rear view of the connector of FIG. 9A;

FIG. 9D is a bottom view of the connector of FIG. 9A;

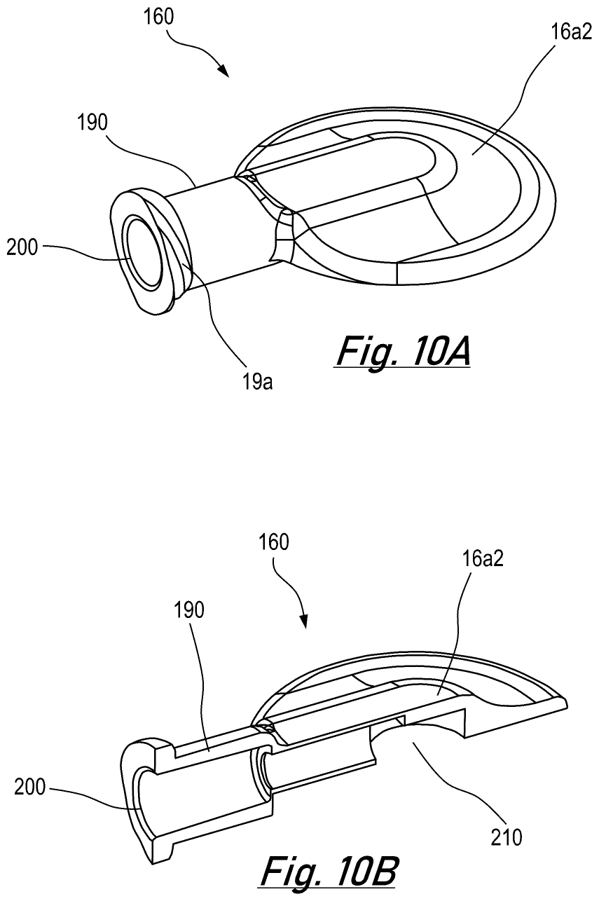

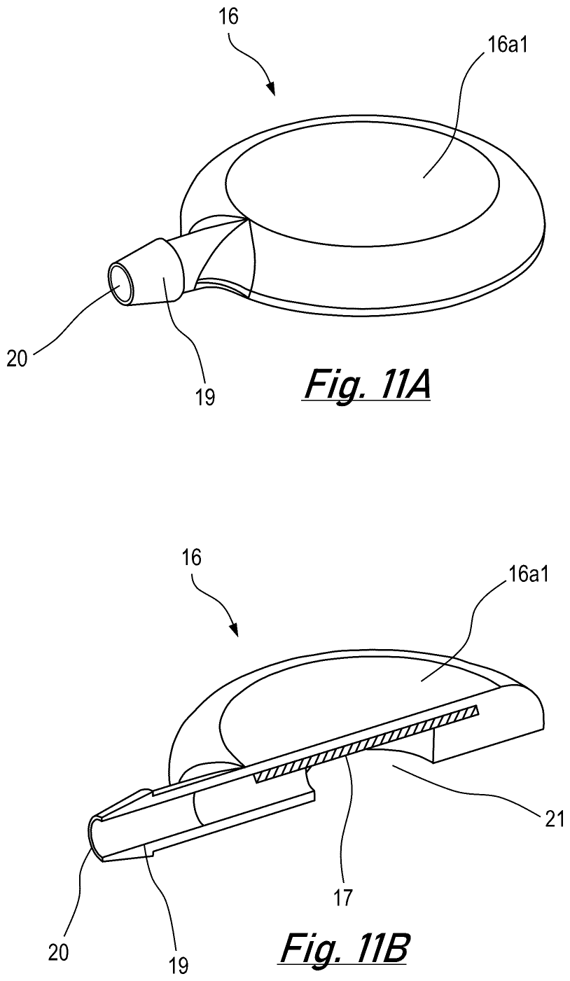

FIG. 10A is a perspective view of the connector of FIG. 9A;

FIG. 10B is a cut away perspective view of the connector of FIG. 9A;

FIG. 11A is a perspective view of the connector of FIG. 8A;

FIG. 11B is a cut away perspective view of the connector of FIG. 8A;

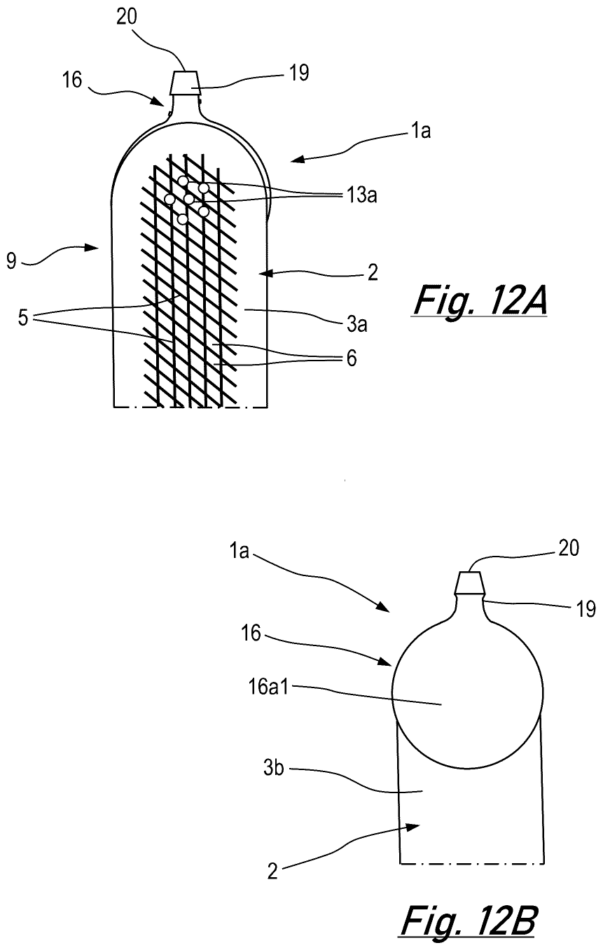

FIG. 12A is part of a plan view of part of an airway according to a further embodiment comprising the connector of FIG. 8A;

FIG. 12B is part of a rear view of the part of the airway of FIG. 12A;

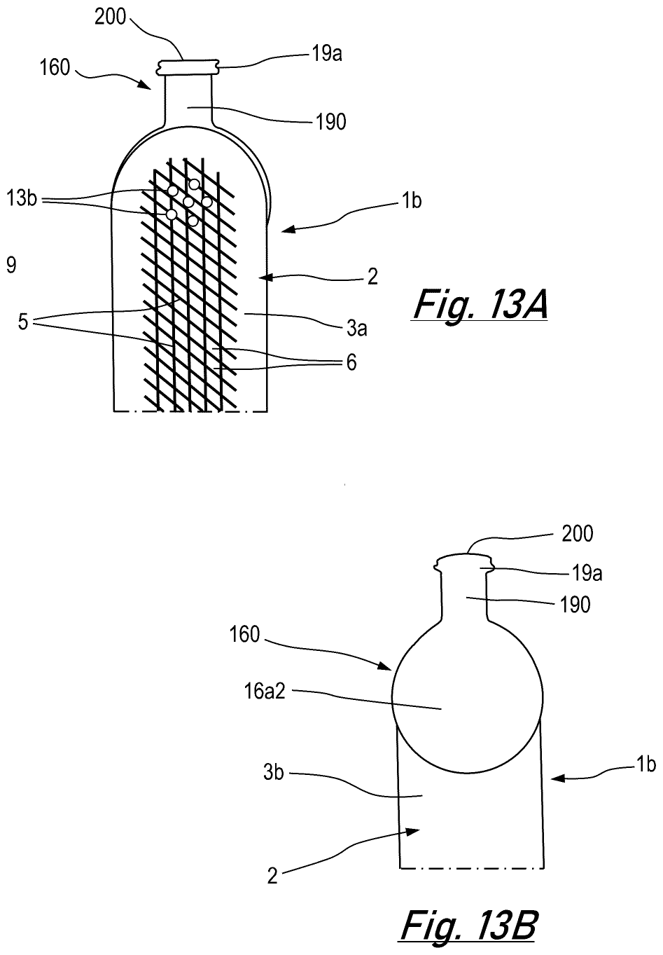

FIG. 13A is part of a plan view of part of an airway according to a further embodiment comprising the connector of FIG. 9A;

FIG. 13B is part of a rear view of the part of airway of FIG. 13A;

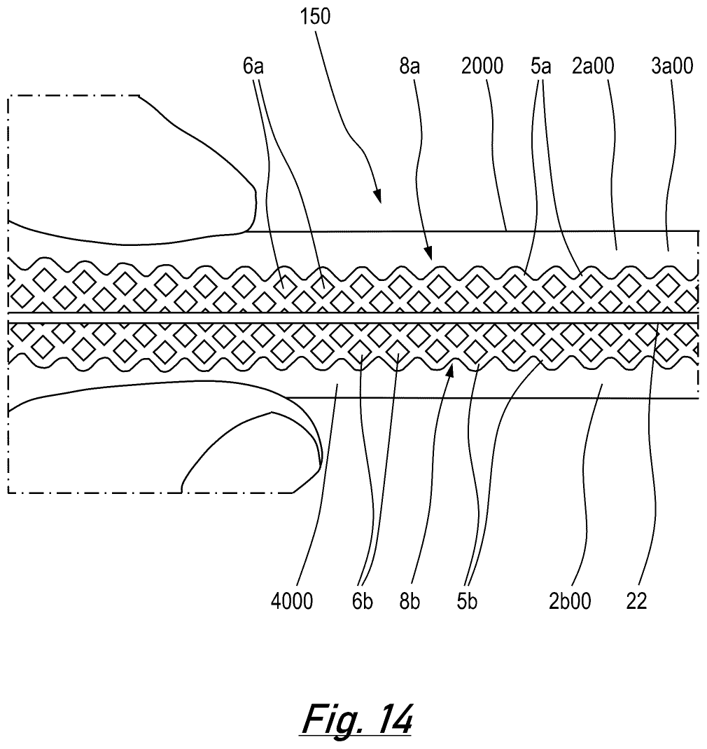

FIG. 14 is a plan view of a third embodiment of an airway;

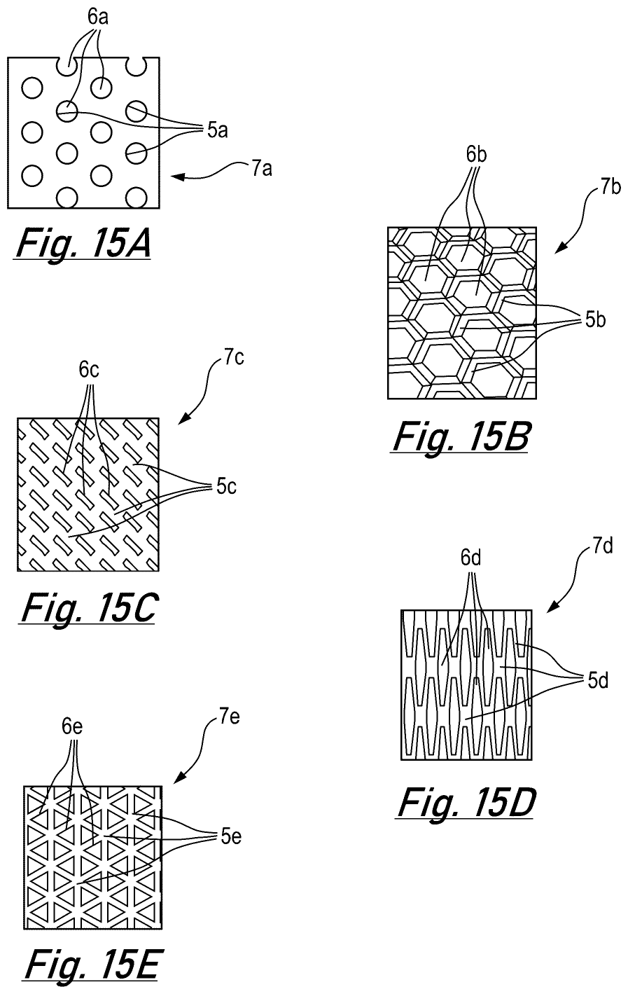

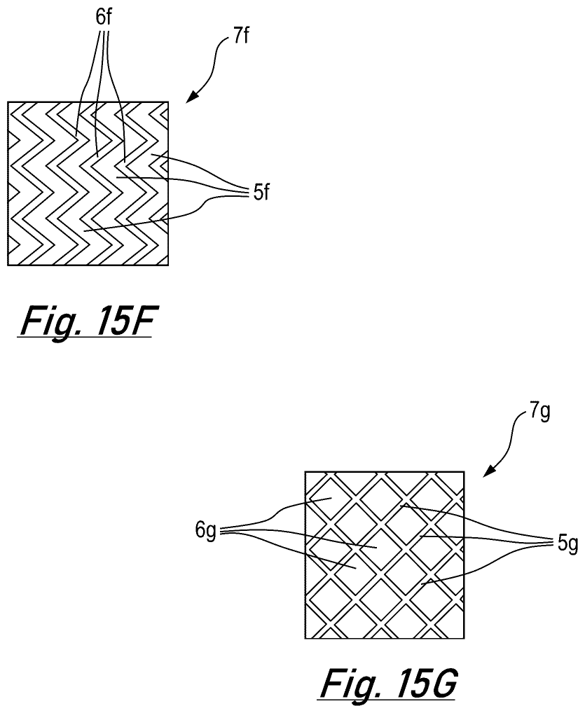

FIG. 15A is a plan view of an example of a plurality of grooves to be used as part of the airway of FIG. 1;

FIG. 15B is a plan view of a further example of a plurality of grooves to be used as part of the airway of FIG. 1;

FIG. 15C is a plan view of a further example of a plurality of grooves to be used as part of the airway of FIG. 1;

FIG. 15D is a plan view of a further example of a plurality of grooves to be used as part of the airway of FIG. 1;

FIG. 15E is a plan view of a further example of a plurality of grooves to be used as part of the airway of FIG. 1;

FIG. 15F is a plan view of a further example of a plurality of grooves to be used as part of the airway of FIG. 1;

FIG. 15G is a plan view of a further example of a plurality of grooves to be used as part of the airway of FIG. 1;

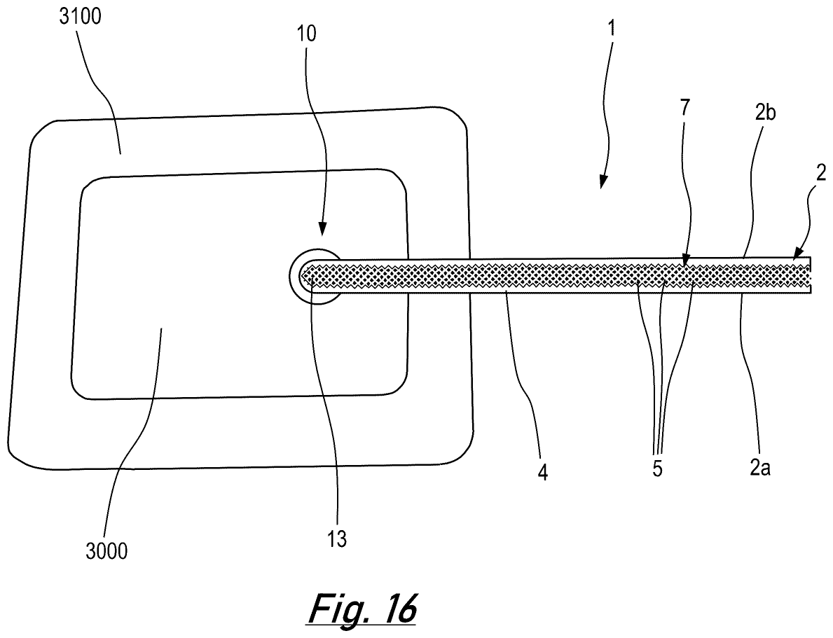

FIG. 16 is a plan view of part of a negative pressure wound dressing comprising the airway of FIG. 1;

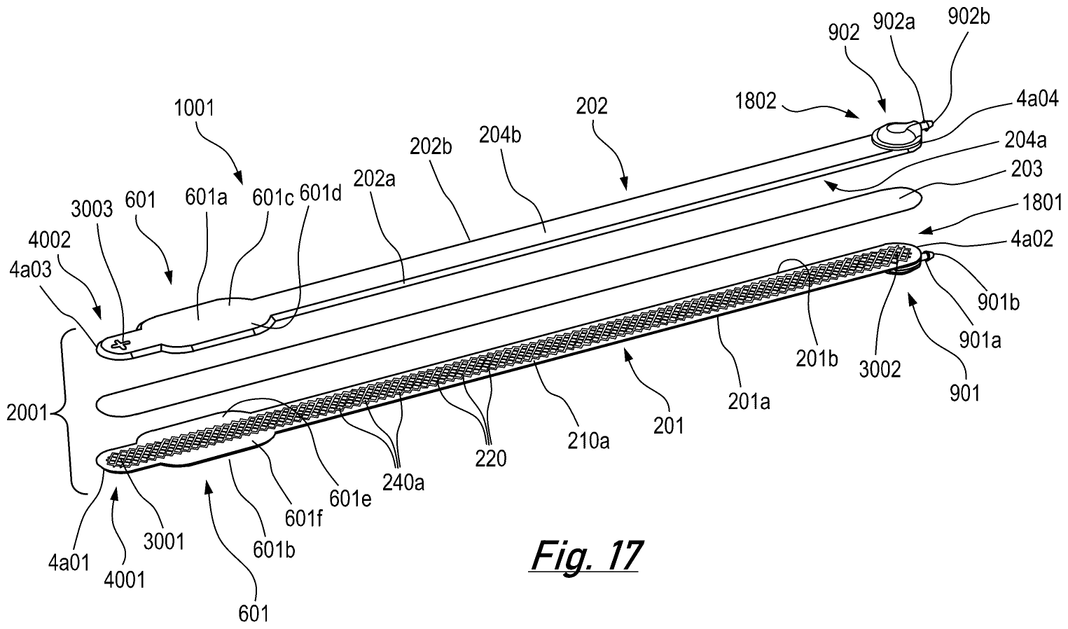

FIG. 17 is an exploded perspective view of an airway according to an even further embodiment;

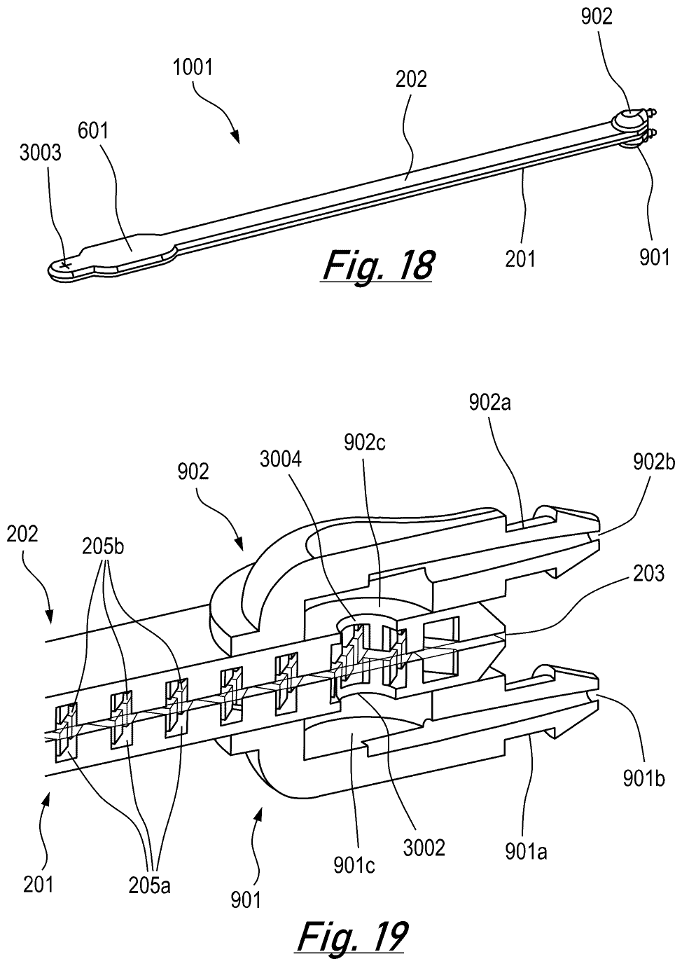

FIG. 18 is a perspective view of the airway shown in FIG. 17;

FIG. 19 is a perspective view of a cut-away of part of the airway shown in FIG. 18;

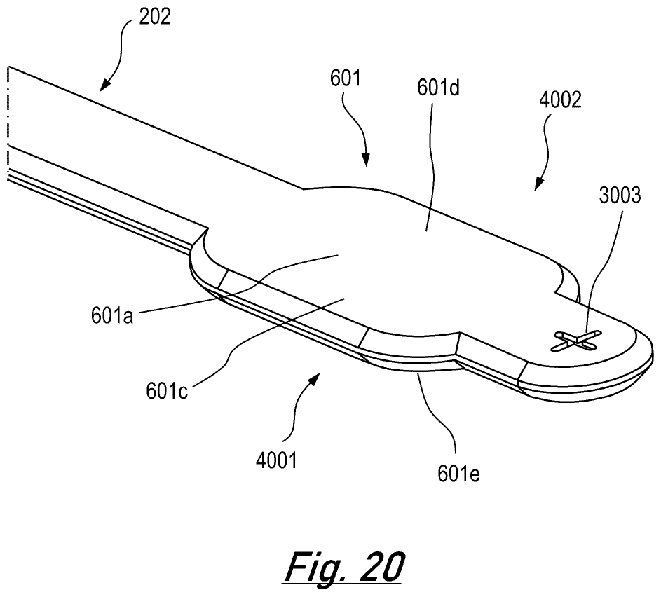

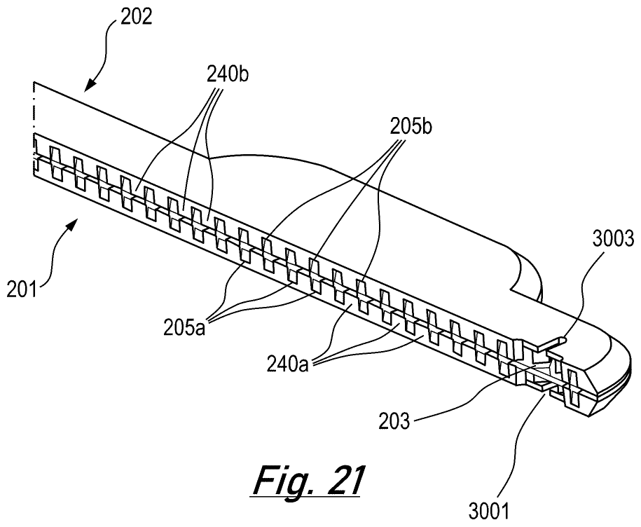

FIG. 20 is a perspective view of part of the airway of FIG. 18; and

FIG. 21 is a perspective view of a cut-away of part of the airway shown in FIG. 18.

Referring to FIGS. 1-7, an embodiment of an airway 1 is shown.

The airway 1 may comprise a strip of material 2 in the shape of an elongated rectangle comprising a first longitudinal edge 2a arranged parallel to a second longitudinal edge 2b. Longitudinal edges 2a, 2b may be joined by an inwardly tapered portion 11 at a first end 9 and by a curved portion at a second end 10. The strip of material 2 may have a width (i.e., the distance between the first longitudinal edge 2a and the second longitudinal edge 2b) of about 15 mm and a length (i.e., the distance from the curved portion of the second end 10 to the terminus of the tapered portion 11) of about 300 mm. The strip of material 2 may comprise an upwardly facing first surface 3a and a downwardly facing second surface 3b. In use, the first surface 3a may face away from the wound site (not shown) and the second surface 3b may face toward the wound site.

The strip of material 2 may be formed of translucent thermoplastic polyurethane, and may be formed by injection moulding.

The first surface 3a of the strip of material 2 may comprise a grooved area 7 comprising a plurality of grooves 5 and a plurality of projections 6. The grooved area 7 may have a width which is equal to about 60% of the width of the strip of material 2, and a length which is equal to about 90% of the length of the strip of material 2. The grooved area 7 may be substantially rectangular. At the first end 9 of the strip of material 2, the grooved area may terminate at substantially the same point at which the strip 2 begins to taper to form the tapered portion 11.

The plurality of grooves 5 may be arranged as an interlaced pattern of criss-crossed grooves 5, with each groove 5 being separated from another groove 5, or another portion of a groove 5, by a projection 6. Each projection 6 may extend from the base of a groove 5. Each groove may have a width of 1 mm and a depth of 1.5 mm. By ‘width’ of a groove, we mean the distance between adjacent projections 6, between which is a groove 5. By ‘depth’ of a groove, we mean the distance between the uppermost surface or point of a projection 6 and the base of the groove 5 from which the projection 6 extends. In the embodiment shown in FIG. 1, each groove 5 has the same width and depth. The grooves may be formed by laser engraving/etching of the strip of material 2. The strip of material 2 may have a depth of 2.0 mm.

Each projection may be arranged between each criss-crossed groove 5. In this embodiment, the projections 6 have a square cross-section when viewed in plan view.

The plurality of grooves 5 allow for the passage of air or another fluid along the airway 1. Each groove 5 is in fluid communication with each other groove such that the plurality of grooves 5 forms an interconnected lattice of grooves in fluid communication with one another. Advantageously, the plurality of grooves 5 of this embodiment increases the overall volume of the grooves 5 of the airway 1. In turn, this means that a greater volume of fluid (for example air) is transmitted along the airway 1 at any given time compared to airways of the prior art which do not comprise a plurality of grooves, in particular an interlaced pattern of grooves. Further, when in use, the interconnected lattice of grooves 5 maintains a favourable flow rate of fluid along the airway 1 during compression or load upon the airway 1. As such, for example when used for transmitting a negative pressure to a wound site, the airway 1 of the invention maintains a negative pressure at a wound site even when the airway 1 has been folded and/or compressed by a user.

The tapered portion 11 may comprise a tubular portion 12. The tubular portion 12 may extend away from the first end 9 to a greater extent than that of the tapered portion 11. The tubular portion 12 may comprise an orifice 12a for receiving a Luer or barb connector (not shown) which is connectable to a pump (not shown) (typically via a section of medical tubing, not shown). The tubular portion 12 may have a depth of 0.8 mm. As such, the tubular portion 12 may be more rigid than any other portion of the airway 1 which facilitates insertion of the Luer or barb connector. The tubular portion 12 may extend for a length equal to less than 10%, for example about 6%, of the length of the airway 1. As shown in FIG. 6, the tubular portion 12 may extend into the airway 1 beyond that at which the strip of material 2 begins to taper. At least one groove 5 is in fluid communication with the tubular portion 12. As such, in use as part of a negative pressure wound dressing, when a pump (not shown) is connected to the tubular portion 12 (for example via a Luer or barb connector), and air is drawn toward the pump, the passage of air flows from the wound site, along the grooves 5 and through the tubular portion 12 toward the pump. As shown in FIGS. 1, 2 and 6 at least, each groove 5 is in fluid communication with each other groove 5. As such, the tubular portion 12 need only be in fluid communication with at least one groove in order to affect the passage of air along all grooves 5.

At the second end 10 of the strip of material 2, the longitudinal edges 2a, 2b may be joined by an outwardly curved end. The second end 10 may comprise a lip 3c which provides a greater surface area of the second surface 3b of the second end 10 for attachment of a double-sided adhesive 14 to the second surface 3b. The double-sided adhesive 14 may comprise a peelable sticker 15 on its wound facing side to protect the adhesive prior to use.

The second end 10 may comprise an aperture 13 in the shape of a cross. The aperture 13 may be arranged centrally relative to the periphery of the second end 10. The double-sided adhesive 14 may comprise an aperture 14a, also in the shape of a cross, arranged concentrically with the aperture 13 of the second end 10.

A cover in the form of a film 4 may be adhered to the first surface 3a of the strip of material 2. The film 4 may have substantially the same width and length as that of the strip of material 2 to prevent overlapping edges of either the strip of material 2 or the film 4. The film 4 may be adhered to the strip of material 2 around its longitudinal edges 2a, 2b. The film 4 may be adhered to the strip of material 2 by heat lamination. The film 4 may be formed of transparent polyurethane. The film 4 may have a thickness of 0.2 mm.

Once the film 4 is adhered to the strip of material 2, the plurality of grooves 5 form a plurality of channels 8 (see FIG. 7) between the strip 2 and the film 4. Each channel of the plurality of channels 8 may be in fluid communication with each other channel such that the plurality of channels 8 forms a network of interconnected channels in fluid communication with one another. The interconnected channels 8 may be in fluid communication with orifice 12a of the tubular portion 12 at the first end 9, and with aperture 13 at the second end 10. The plurality of channels 8 allow for the passage of air to or from the wound site via a pump, for example used as part of a negative pressure wound dressing system. Compression of the film 4 toward the strip of material 2 is prevented, or at least significantly limited, by the plurality of projections 6 arranged along the grooved area 7. Compression of the film 4 toward the strip of material 2 is also prevented, or at least significantly limited, by the relatively small width of the plurality of grooves 5 such that when a typical load exerts pressure upon the film 4, the film 4 does not compress into the grooves 5, or at least compresses into a portion of the grooves 5 only, so that the flow of fluid along the grooves 5 is not prevented. In use, when a negative pressure is applied to the wound site and air is drawn away from the wound site along the plurality of channels 8, the film 4 will compress downwardly, toward the channels 8. However, obstruction of the channels 8 by the film 4 is prevented by the plurality of projections 6 upon which the film 4 compresses. As such, the film 4 does not collapse into the channels 8, therefore, the present arrangement allows for the unobstructed passage of air along the channels 8.

The film 4 may be substantially planar.

It will be understood that the dimensions (depth, width and length) of each channel 8 will be substantially the same as the corresponding dimensions of each groove 5 because adhesion of the film 4 onto the first surface 3a of the strip of material 3 prevents compression of the film 4 into each groove 5.

As an example of the present embodiment in use, a typical negative pressure wound dressing (shown in part in FIG. 16) comprises a pump (not shown) for generating negative pressure and a dressing 3000 for covering and protecting a wound. The dressing comprising a wound contact layer, a backing layer comprising a peripheral adhesive 3100 for adhering the wound dressing 3000 to the skin of a user, and at least one layer of absorbent material between the wound contact layer and the backing layer. In use, a fluid communication pathway is provided from the wound, through the wound contact layer and through one or more layers of absorbent material disposed in the dressing. The fluid communication pathway may also extend through an opening in the backing layer. Advantageously, the fluid communication pathway can be formed at any suitable location on the wound dressing. Alternatively, provided the fluid communication pathway extends through an opening in the backing layer, the airway 1 may be adhered to the wound dressing on the backing layer. The aperture 13 in the second end 10 of the strip of material 2 and the aperture 14a in the adhesive panel 14 are arranged concentrically with the fluid communication pathway. A Luer or barb connector is inserted into the tubular portion 12 of the first end 9, and the connector is connected to a pump, possibly via a tube or similar. Activation of the pump draws air from the wound site, through the wound contact layer and absorbent material via the fluid communication pathway, through the aperture 14a of the adhesive panel 14, aperture 13 of the strip of material 2, along the plurality of channels 8, through the tubular portion 12 and Luer or barb connector and toward the pump. Thus, a negative pressure is created at the wound site.

FIGS. 8A-D show an example of a connector 16 for use with an airway 1a according to a further embodiment which may not comprise a tubular portion 12 (described further below). The connector 16 may comprise an upper surface 16a1 and a lower surface 16b1 joined at their periphery. The connector 16 may be substantially round except for a nozzle 19 which protrudes from the periphery where the upper surface 16a1 and lower surface 16b1 join. The nozzle 19 may be about 10 mm in length and may protrude outwardly, away from the connector 16, and may comprise a mouth 20 at its distal end. The mouth 20 may be in fluid communication with the nozzle 19 and the interior of the connector 16. The lower surface 16b1 may comprise a circular orifice 21 arranged central relative to the periphery of the lower surface 16b1. The orifice 21 may be in fluid communication with the nozzle 19 and the mouth 20.

FIGS. 9A-D and 10A-B show an example of a connector 160 for use with an airway 1b which may not comprise a tubular portion 12. The connector 160 may comprise an upper surface 16a2 and a lower surface 16b2 joined at their periphery. The connector 160 may be substantially round except for a nozzle 190 which protrudes from the periphery where the upper surface 16a2 and lower surface 16b2 join. The nozzle 190 may be about 10 mm in length and may protrude outwardly, away from the connector 160, and may comprise a mouth 200 at its distal end. The nozzle 190 may comprise a threaded end 19a surrounding the mouth 200. Thus, the connector 160 can be connected to a Luer or barb connector, or similar, which comprises a corresponding threaded end for secure connection. The mouth 200 may be in fluid communication with the nozzle 190 and the interior of the connector 160. The lower surface 16b2 may comprise an orifice 210 arranged central relative to the periphery of the lower surface 16b2. The orifice 210 may be in fluid communication with the nozzle 190 and the mouth 200.

FIGS. 11A-B show an example embodiment of the connector 16 comprising a filter disk 17 located within the nozzle 19. The filter disk 17 may be impermeable to liquids but permeable to gases and therefore can function as a liquid barrier to ensure that no wound exudate can exit the wound dressing.

The filter disk 17 may also function as a bacterial barrier.

The filter disk 17 may comprise a microporous hydrophobic membrane formed from polypropylene. The filter disk 17 may also comprise activated charcoal as an odour absorbent material.

The filter disk 17 may comprise a plurality of pores having an average pore size of 0.5 km.

Whilst it is not shown in the described embodiment, the filter disk 17 may further comprise an infection detection indicator and/or a dressing change indicator.

In embodiments shown in FIGS. 12A, 12B, 13A and 13B, the airway 1a, 1b may not comprise a tapered portion or a tubular portion at its first end. In such embodiments, the airway 1a, 1b may comprise at least one aperture 13a, 13b through the strip of material 2 at its first end 9. In these embodiments, a connector 16, 160 may be arranged such that the orifice 21, 210 is arranged in fluid communication with the aperture at the first end. The connector 16, 160 may be adhered (by any suitable means, for example using an adhesive) to the second surface 3b of the airway 1a, 1b so as to not interfere with the film adhered to the first surface. The nozzle 19, 190 may be connected to a pump via a suitable connector, for example a Luer or barb connector, by insertion of the nozzle into the Luer or barb connector, or by insertion of the Luer or barb connector into the nozzle 19, 190, as appropriate.

FIGS. 12A and 12B show the connector 16 adhered to the airway 1a. Airway 1a may comprise a first end 9 having a rounded end to join longitudinal edges of the airway 1a. The connector 16 is adhered to the second surface 3b of the airway 1a at its first end 9. The airway 1a may comprise a plurality of apertures 13a at its first end 9. In the described embodiment, the first end 9 comprises six apertures 13a. Each aperture 13a may be circular and may have a diameter of about 2 mm. The circular orifice 21 may be in fluid communication with the plurality of apertures 13a. In embodiments where the airway 1a is used as part of a negative pressure wound dressing, one end of medical tubing (not shown) may be friction fitted around nozzle 19, and the other end of the medical tubing may be connected to a pump. The remaining features of airway 1b are much the same as those described in connection with the embodiment shown in FIG. 1.

FIGS. 13A and 13B show the connector 160 adhered to the airway 1b. Airway 1b may comprise a first end 9 having a rounded end to join longitudinal edges of the airway 1b. The connector 160 is adhered to the second surface 3b of the airway 1b at its first end 9. The airway 1b may comprise a plurality of apertures 13b at its first end 9. In the described embodiment, the first end 9 comprises six apertures 13b. Each aperture 13b may be circular and may have a diameter of about 2 mm. The circular orifice 210 may be in fluid communication with the plurality of apertures 13b. In embodiments where the airway 1b is used as part of a negative pressure wound dressing, one end of medical tubing (not shown) may be friction fitted around nozzle 19a, and the other end of the medical tubing may be connected to a pump.

FIG. 14 shows an airway 150 according to a third embodiment.

Airway 150 may comprise an elongated strip of material 2000 comprising longitudinal edges 2a00, 2b00, and a first surface 3a00 and a second surface (not shown). Many of the features of airway 150 are identical to that of airway 1 and airway 100. However, airway 150 differs from airway 1 in that airway 150 may comprise two distinct, separated channelled areas 8a, 8b. The airway 150 may comprise a first channelled area 8a and a second channelled area 8b. The first channelled area 8a may be separated from the second channelled area 8b by a seal formed between the film 4000 and the strip of material 2000 after or simultaneously with adhering the film 4000 to the strip of material 2000. The first channelled area 8a may not be in fluid communication with the second channelled area 8b. The seal may be formed by heat lamination. The first channelled area 8a may comprise two criss-crossing channels, and the second channelled area 8b may comprise two criss-crossing channels. Each channelled area 8a, 8b may be formed by the combination of grooves and projections as in the previously mentioned embodiments. Advantageously, in this embodiment, fluid can flow toward a wound site via the channels in the first channelled area 8a, and fluid can simultaneously flow away from a wound site via the channels in the second channelled area 8b.

FIGS. 15A-15G show different embodiments of a grooved area 7. FIG. 15A shows a grooved area 7a comprising a plurality of grooves 5a and a plurality of discrete, individual projections 6a. Each of the projections 6a have a circular cross section. FIG. 15B shows a grooved area 7b comprising a plurality of grooves 5b and a plurality of discrete, individual projections 6b. Each of the projections 6b have a hexagonal cross section. FIG. 15C shows a grooved area 7c comprising a plurality of grooves 5c and a plurality of discrete, individual projections 6c. Each of the projections 6c have a rectangular cross section. FIG. 15D shows a grooved area 7d comprising a plurality of grooves 5d and a plurality of discrete, individual projections 6d. Each of the projections 6d have a rhombus cross section. FIG. 15E shows a grooved area 7e comprising a plurality of grooves 5e and a plurality of discrete, individual projections 6e. Each of the projections 6e have a triangular cross section. FIG. 15F shows a grooved area 7f comprising a plurality of grooves 5f and a plurality of projections 6f. Each of the projections 6f are in the form of linear zig zags. FIG. 15G shows a grooved area 7g comprising a plurality of grooves 5g and a plurality of discrete, individual projections 6g. Each of the projections 6g have a square cross section. Each groove may have a width of 0.5 mm and a depth of 1.5 mm.

Each grooved area, 7a, 7b, 7c, 7d, 7e, 7e, 7g shown in each of FIGS. 15A-15G can equally be used as part of the airway shown in FIG. 1, 12 or 13.

With reference to FIGS. 17 to 21, there is shown an airway 2001 of a further embodiment of the invention. The airway 2001 may be used as an adaptor 1001 for adapting a surgical wound dressing for use in negative pressure wound therapy. The airway 2001 may be an elongate, flexible airway 2001. The airway 2001 may be formed of a liquid impermeable material, for example a material selected from the group comprising polyurethane, thermoplastic polyurethane, thermoplastic elastomer, polyethylene, polypropylene, polyvinyl chloride, ethylene-vinyl acetate and a silicone material. In the described embodiment, the airway 2001 is formed of translucent thermoplastic polyurethane, and may be formed by injection moulding.

The airway 2001 may have a maximum width which is greater than the maximum depth of the airway 2001. The airway 2001 may have a maximum depth of from 2.0 mm to 5.0 mm. In the described embodiment, the airway 2001 has a maximum depth of 3.5 mm. In the described embodiment, the airway 2001 has a maximum width which is at least two times the maximum depth of the airway 2001.

The airway 2001 may comprise a first elongated strip of material 201 comprising a pair of longitudinal edges 201a, 201b.

The first elongated strip of material 201 may comprise a first end 1801 and a second end 4001. The longitudinal edges 201a, 201b may be joined at the first end 1801 by an outwardly curved portion, the outermost part of which forms the terminus 4a02 of the first end. The longitudinal edges 201a, 201b may be joined by an outwardly curved portion, the outermost part of which forms the terminus 4a01 of the second end 4001. The second end 4001 may be distal to the first end 1801. The strip of material 201 may have a width (i.e., the distance between the first longitudinal edge 201a and the second longitudinal edge 201b) of about 15 mm and a length (i.e., the distance from the terminus 4a02 of the first end 1801 to the terminus 4a01 of the second end 4001) of about 300 mm.

The first end 1801 may comprise a first orifice 3002 in the shape of a cross. The first orifice 3002 may be arranged central relative to the periphery of the first end 1801.

The strip of material 201 may comprise an upwardly facing first surface 210a and a downwardly facing second surface. In use, the first surface 210a may face away from the wound site (not shown) and the second surface may face toward the wound site.

The first surface 210a of the strip of material 201 may comprise a grooved area comprising a plurality of grooves 220 and a plurality of projections 240a. The grooved area may have a width which is equal to about 60% of the width of the strip of material 201, and a length which is equal to about 90% of the length of the strip of material 201. The grooved area may be substantially rectangular.

The plurality of grooves 220 may be arranged as an interlaced pattern of criss-crossed grooves 220, with each groove 220 being separated from another groove 220, or another portion of a groove 220, by a projection 240a. Each projection 240a may extend from the base of a groove 220. Each groove 220 may have a width of 1 mm and a depth of 1.5 mm. The grooves may be formed by laser engraving/etching of the strip of material 201. The strip of material 201 may have a depth of 2.0 mm.

The plurality of grooves 220 allows for the passage of air or another fluid along the airway 2001. Each groove 220 may be in fluid communication with each other groove 220 such that the plurality of grooves 220 forms an interconnected lattice of grooves in fluid communication with one another. Advantageously, the plurality of grooves 220 in this embodiment increases the overall volume of the grooves 220 of the airway 2001. In turn, this means that a greater volume of fluid (for example air) is transmitted along the airway 2001 at any given time compared to known airways of the prior art which do not comprise a plurality of grooves, in particular an interlaced pattern of grooves. Further, when in use, the interconnected lattice of grooves 220 maintains a favourable flow rate of fluid along the airway 2001 during compression or load upon the airway 2001. As such, for example when used for transmitting a negative pressure to a wound site, the airway 2001 of the invention maintains a negative pressure at a wound site even when the airway 2001 has been folded and/or compressed by a user.

The second end 4001 of the first strip of material 201 may comprise a second orifice 3001 in the shape of a cross. The second orifice 3001 may be arranged central relative to the periphery of the second end 4001.

At least one groove 220 may be in fluid communication with the first orifice 3002 at the first end 1801, and with the second orifice 3001 at the second end 4001.

The airway 2001 may comprise a film 203. The film 203 may be adhered to the first surface 210a of the first strip of material 201. The film 203 may have substantially the same width and length as that of the strip of material 201 to prevent overlapping edges of either the strip of material 201 or the film 203. The film 203 may be adhered to the strip of material 201 at its longitudinal edges. The film 203 may be adhered to the strip of material 201 by heat lamination. The film 203 may be formed of transparent or translucent polyurethane. The film 203 may have a thickness of about 0.2 mm.

Once the film 203 is adhered to the first strip of material 201, the plurality of grooves 220 form a first plurality of channels 205a between the first strip of material 201 and the film 203. Each channel of the first plurality of channels 205a may be in fluid communication with each other channel such that the first plurality of channels 205a forms a network of interconnected channels in fluid communication with one another. The interconnected channels 205a may be in fluid communication with the first orifice 3002 at the first end 1801 and with the second orifice 3001 at the second end 4001.

The first plurality of channels 205a allow for the passage of air to or from the wound site via a pump, for example used as part of a negative pressure wound dressing system. Compression of the film 203 toward the strip of material 201 may be prevented, or at least significantly limited, by the plurality of projections 240a arranged along the grooved area.

Compression of the film 203 toward the strip of material 201 may also be prevented, or at least significantly limited, by the relatively small width of the plurality of grooves 220 such that when a typical load exerts pressure upon the film 203, the film 203 does not compress into the grooves 220 so that the flow of fluid along the grooves 220 is not prevented.

The film 203 may be substantially planar.

The airway 2001 may comprise a second strip of material 202. The second strip of material 202 may be elongated.

The second strip of material 202 may comprise a first end 1802 and a second end 4002. The second elongated strip of material 202 may comprise a pair of longitudinal edges 202a, 202b. The longitudinal edges 202a, 202b may be joined at the first end 1802 by an outwardly curved portion, the outermost part of which forms the terminus 4a04 of the first end. The longitudinal edges 202a, 202b may be joined by an outwardly curved portion, the outermost part of which forms the terminus 4a03 of the second end 4002. The second end 4002 may be distal to the first end 1802. The second strip of material 202 may have a width (i.e., the distance between the first longitudinal edge 202a and the second longitudinal edge 202b) of about 15 mm and a length (i.e., the distance from the terminus 4a04 of the first end 1802 to the terminus 4a03 of the second end 4002) of about 300 mm.

The first end 1802 of the second strip of material 202 may comprise a third orifice 3004 in the shape of a cross. The third orifice 3004 may be arranged central relative to the periphery of the first end 1802.

The second strip of material 202 may comprise a downwardly facing first surface 204a and an upwardly facing second surface 204b. In use, the first surface 204a may face toward the wound site (not shown) and the second surface 204b may face away from the wound site.

The first surface 204a of the second strip of material 202 may comprise a grooved area comprising a plurality of grooves (not shown) and a plurality of projections 240b. The grooved area of the first surface 204a of the second strip of material 202 may comprise each of the features of the grooved area of the first surface 210a of the first strip of material 201. The grooved area may have a width which is equal to about 60% of the width of the second strip of material 202, and a length which is equal to about 90% of the length of the second strip of material 202. The grooved area may be substantially rectangular.

The plurality of grooves may be arranged as an interlaced pattern of criss-crossed grooves, with each groove being separated from another groove, or another portion of a groove, by a projection 240b. Each projection 240b may extend from the base of a groove. Each groove may have a width of 1 mm and a depth of 1.5 mm. The grooves may be formed by laser engraving/etching of the strip of material 202. The strip of material 202 may have a depth of 2.0 mm.

Each groove may be in fluid communication with each other groove such that the plurality of grooves forms an interconnected lattice of grooves in fluid communication with one another. Advantageously, the plurality of grooves in this embodiment increases the overall volume of the grooves of the airway 2001. In turn, this means that a greater volume of fluid (for example air) is transmitted along the airway 2001 at any given time compared to airways of the prior art which do not comprise a plurality of grooves, in particular an interlaced pattern of grooves. Further, when in use, the interconnected lattice of grooves maintains a favourable flow rate of fluid along the airway 2001 during compression or load upon the airway 2001. As such, for example when used for transmitting a negative pressure to a wound site, the airway 2001 of the invention maintains a negative pressure at a wound site even when the airway 2001 has been folded and/or compressed by a user.

The second end 4002 may comprise a fourth orifice 3003 in the shape of a cross. The fourth orifice 3003 may be arranged central relative to the periphery of the second end 4002.

At least one groove may be in fluid communication with the third orifice 3004 at the first end 1802 of the second strip of material 202, and with the fourth orifice 3003 at the second end 4002 of the second strip of material 202.

The airway 2001 may comprise the second strip of material 202 such that first surface 204a of the second strip 202 is arranged over the film 203. The first surface 204a of the second strip 202 may be adhered to the film 203 (for example by an adhesive, alternatively heat lamination may be used) about its longitudinal edges 202a, 202b. Once the second strip of material 202 is adhered to the film 203, the plurality of grooves form a second plurality of channels 205b between the second strip of material 202 and the film 203. Each channel of the second plurality of channels 205b may be in fluid communication with each other channel such that the second plurality of channels 205b forms a network of interconnected channels in fluid communication with one another. The interconnected channels 205b may be in fluid communication with the third orifice 3004 at the first end 1802 and with the fourth orifice 3003 at the second end 4002.

The peripheral dimensions of the second strip of material 202 may be substantially equal to the peripheral dimensions of the first strip of material 201.

The plurality of channels 205b may allow for the passage of air to or from the wound site via a pump, for example used as part of a negative pressure wound dressing system. Compression of the film 203 toward the strip of material 202 may be prevented, or at least significantly limited, by the plurality of projections 240b arranged along the grooved area.

Compression of the film 203 toward the strip of material 202 is also prevented, or at least significantly limited, by the relatively small width of the plurality of grooves such that when a typical load exerts pressure upon the film 203, the film 203 does not compress into the grooves, or at least compresses into a portion of the grooves only, so that the flow of fluid along the grooves is not prevented.

In the described embodiment, the first plurality of channels 205a formed between the first strip of material 201 and the film 203 are not in fluid communication with the second plurality of channels 205b formed between the second strip of material 202 and the film 203.

The first orifice 3002 comprised in the first strip 201 and the third orifice 3004 comprised in the second strip 202 may not be in fluid communication with one another, and may be separated from one another by the film 203.

The second orifice 3001 comprised in the first strip 201 and the fourth orifice 3003 comprised in the second strip 202 may not be in fluid communication with one another, and may be separated from one another by the film 203.

In some embodiments, the airway 2001 may comprise a sealing portion 601. The airway 2001 may comprise the sealing portion 601 toward its second end 4001, 4002 but distal to the terminus 4a01, 4a03 of the second end 4001, 4002 relative to the location of the orifices 3001, 3003. As such, the orifices 3001, 3003 may be arranged between the terminus 4a01, 4a03 of the second end 4001, 4002 and the sealing portion 601. The length of the airway 2001 from its terminus 4a01, 4a03 at the second end 4001, 4002 to the sealing portion 601 may be no more than about 5 cm. In the described embodiment, the length of the airway 2001 from its terminus 4a01, 4a03 to the sealing portion 601 is about 2 cm.

In use, the sealing portion 601 provides a surface for adhering the adaptor 1001 to an underside of a surgical wound dressing, such as to a peripheral adhesive skin contact layer of a surgical wound dressing. The sealing portion 601 may provide a surface for adhering the adaptor 1001 to the peri wound skin of a patient.

The sealing portion 601 may provide a surface for adhering the airway 2001 to a backing layer of a wound dressing, such that, in use, a fluid communication pathway is provided from the wound, through the wound contact layer and through one or more layers of absorbent material disposed in the dressing. The fluid communication pathway may also extend through an opening in the backing layer. Advantageously, the fluid communication pathway can be formed at any suitable location on the wound dressing.

The sealing portion 601 may comprise an upper sealing surface 601a and a lower sealing surface 601b. In use, the upper sealing surface 601a may provide the surface for adhering the adaptor 1001 to an underside of a surgical wound dressing, such as to a peripheral adhesive skin contact layer of a surgical wound dressing. In use, the lower sealing surface 601b may provide the surface for adhering the adaptor 1001 to the peri wound skin of a patient. As such, the sealing portion 601 of the adaptor 1001 is configured to provide a complete peripheral seal between the surgical wound dressing and the patient.

The lower sealing surface 601b may comprise an adhesive layer (not shown) which, prior to use, may be covered by a removable release liner (not shown). The removable release liner may prevent accidental exposure of the adhesive layer prior to use which may otherwise be detrimental to the adhesive property of the adhesive layer.

The upper sealing surface 601a may comprise a first wing 601c and a second wing 601d. The lower sealing surface 601b may comprise a third wing 601e and a fourth wing 601f. Each wing 601c, 601d, 601e, 601f may extend outwardly from the airway 2001 in a direction perpendicular to the length of the airway 2001. Each wing 601c, 601d, 601e, 601f may extend outwardly from its respective longitudinal edge of the airway 2001 a distance of about 1.5 cm, such that the width of the sealing portion 601, that is the length from the outermost point of the first wing 601c and third wing 601e to the outermost point of the second wing 601d and fourth wing 601f, may be about 5 cm. Each wing curves outwardly, from its respective longitudinal edge of the airway 2001, to a distance of about 1.5 cm from the longitudinal edge, and then extends linearly to the length of the airway 2001, such that the linear portion of the first wing 601c is parallel with the linear portion of the second wing 601d, and the linear portion of the third wing 601e is parallel with the linear portion of the fourth wing 601f, for a distance of about 3 cm, and then each wing curves toward the respective longitudinal edge of the airway 2001.

An upper surface of the first wing 601c and upper surface of the second wing 601d may each comprise a portion of the upper sealing surface 601a. A lower surface of the third wing 601e and a lower surface of the fourth wing 601f may each comprise a portion of the lower sealing surface 601b. The upper sealing surface 601a may be substantially planar to provide a substantially flat upper surface to the airway 2001. The lower sealing surface 601b may be substantially planar to provide a substantially flat lower surface to the airway 2001.

The first end 1801 of the first strip of material 201 may comprise a first connector 901 adhered to its second surface. The first connector 901 may comprise a nozzle 901a having an aperture 901b, and a surface comprising an orifice 901c arranged in fluid communication with the aperture 901b. The first connector 901 may be arranged such that the orifice 901c is in fluid communication with the first orifice 3002 at the first end 1801 of the first strip 201. Thus, the first plurality of channels 205a may be in fluid communication with the orifice 901c of the first connector 901, and also, therefore, the aperture of the nozzle 901b.

The first end 1802 of the second strip of material 202 may comprise a second connector 902 adhered to its upwardly facing second surface 204b. The second connector 902 may comprise a nozzle 902a having an aperture 902b, and a surface comprising an orifice 902c in fluid communication with the aperture 902b. The second connector 902 may be arranged such that the orifice 902c is in fluid communication with the third orifice 3004 at the first end 1802 of the second strip of material 202. Thus, the second plurality of channels 205b may be in fluid communication with the orifice 902c of the second connector 902, and therefore, the aperture of the nozzle 902b.

A pump (not shown) may be connected to one or each of the first and second connectors 901, 902 (for example via a Luer or barb connector and medical tubing).

As such, the airway 2001, or in use as an adaptor 1001 for adapting a surgical wound dressing for use in negative pressure wound therapy, a pump (not shown) may be connected to the first connector 901 (for example via a Luer or barb connector and medical tubing), and air may be drawn toward the pump. The passage of air may flow from the wound site, through the second orifice 3001, along the first plurality of channels 205a and through the first orifice 3002, through the orifice 901c of the first connector 901, along the nozzle 901a, through the aperture 901b, and along any medical tubing toward the pump.

Further, the embodiment shown in FIGS. 17 to 21 allows for a fluid (for example air) to be delivered to the wound site simultaneously to the drawing of air from the wound site, as described above. For example, a pump (not shown) may be connected to the second connector 902 (for example via a Luer or barb connector and medical tubing), and air may be delivered from the pump toward the wound site. The passage of air may flow from the pump, along any medical tubing, through the aperture 902b of the nozzle 902a of the second connector 902, through the orifice 902c of the second connector 902, and through the third orifice 3004, along the second plurality of channels 205b and through the fourth orifice 3003, toward the wound site.

The airway of any of the above-described embodiments may be used with a negative pressure wound dressing or standard wound dressing for the application of negative pressure therapy, positive pressure therapy, oxygen therapy or deep wound filling or other treatments requiring the flow of fluid to and/or from a wound site.

The one or more embodiments are described above by way of example only. Many variations are possible without departing from the scope of protection afforded by the appended claims.

1. An airway for a negative pressure wound dressing, the airway comprising a first strip of material having a first surface comprising at least one groove, the airway further comprising a film arranged over the first surface of the first strip of material and adhered to the first strip of material around its longitudinal edges, wherein the at least one groove is provided with at least one projection to form at least two channels between the first strip and the film, wherein the airway further comprises a second strip of material having a first surface comprising at least one groove provided with at least one projection, the first surface of the second strip of material being arranged over the film and adhered to the film around its longitudinal edges to form at least two channels, between the second strip and the film.

2. An airway according to claim 1, wherein the at least one groove comprised in the first surface of the first strip of material is provided with a plurality of projections, such that there is provided a plurality of channels between the first strip and the film, and/or wherein the at least one groove comprised in the first surface of the second strip of material is provided with a plurality of projections, such that there is provided a plurality of channels between the second strip and the film.

3. An airway according to claim 2, wherein at least two channels between the first strip of material and the film are in fluid communication with one another, and/or wherein at least two channels between the second strip of material and the film are in fluid communication with one another.

4. An airway according to claim 3, wherein the channels between the first strip of material and the film form a network of interconnected channels, and/or wherein the channels between the second strip of material and the film form a network of interconnected channels.

5. An airway according to any preceding claim, wherein the airway comprises a first end comprising an orifice operable as an inlet or outlet for the flow of fluid, and a second end comprising an orifice operable as the other of the inlet or outlet for the flow of fluid.

6. An airway according to claim 5, wherein the orifice of the first end is in fluid communication with the orifice of the second end via the channels between the first strip of material and the film, or via the channels between the second strip of material and the film.

7. An airway according to any of claims 2 to 4, wherein the plurality of channels between the first strip of material and the film is arranged as an interlaced pattern of channels, and/or wherein the plurality of channels between the second strip of material and the film is arranged as an interlaced pattern of channels.

8. An airway according to claim 7, wherein the interlaced pattern of channels between the first strip of material and the film comprises a plurality of criss-crossed channels, and/or wherein the interlaced pattern of channels between the second strip of material and the film comprises a plurality of criss-crossed channels.

9. An airway according to claim 7 or 8, wherein the plurality of channels between the first strip of material and the film is arranged as a zig-zag pattern of channels, and/or wherein the plurality of channels between the second strip of material and the film is arranged as a zig-zag pattern of channels.

10. An airway according to claim 1, wherein the at least two channels between the first strip of material and the film comprises channels arranged parallel to one another, and/or wherein the at least two channels between the second strip of material and the film comprises channels arranged parallel to one another.

11. An airway according to any preceding claim, wherein the or each projection extends from a base of the or each groove.

12. An airway according to any preceding claim, wherein the or each groove form a total grooved area that has a width which is equal to at least 50% of the width of the airway.

13. An airway according to any preceding claim, wherein the at least two channels between the first strip of material and the film comprises at least a first channel and a second channel, wherein the first channel is not in fluid communication with the second channel, and/or wherein the at least two channels between the second strip of material and the film comprises at least a first channel and a second channel, wherein the first channel is not in fluid communication with the second channel.

14. An airway according to claim 13, wherein the first channel and the second channel, each between the first strip of material and the film, are separated by a seal between the strip and the film, and/or wherein the first channel and the second channel, each between the second strip of material and the film, are separated by a seal between the second strip and the film.

15. An airway according to any preceding claim, wherein the film is laminated to the first strip of material and/or to the second strip of material.

16. An airway according to any preceding claim, wherein the airway comprises a first end, the first end comprising a filter, an infection detection indicator and/or a dressing change indicator.

17. An airway according to claim 16, wherein the first end comprises a connector for connecting the airway to a pump.

18. An airway according to claims 16 and 17, wherein a filter, an infection detection indicator and/or a dressing change indicator are formed integral with the connector.

19. An airway according to any of claims 16 to 18, wherein a filter, an infection detection indicator and/or a dressing change indicator and/or a connector is attached to the first surface of the first strip of material.

20. An airway according to any of claims 16 to 18, wherein a filter, an infection detection indicator and/or a dressing change indicator and/or connector is attached to a second surface of the first strip of material and is in fluid connection with the first surface via one or more apertures in the strip of material.

21. An airway according to any of claims 16 to 18, wherein the airway comprises a second end, the second end comprising an adhesive panel for adhering the airway to a wound dressing.

22. An airway according to any preceding claim, wherein the at least two channels between the first strip of material and the film are not in fluid communication with the at least two channels between the second strip of material and the film.

23. An airway according to any of claims 17 to 20, wherein the connector is a first connector for connection of the at least two channels between the first strip of material and the film to a first medical tubing and an interior lumen therebetween.

24. An airway according to claim 5 or 6, wherein the orifice of the first end is a first orifice and the at least two channels between the first strip of material and the film are in fluid communication with the first orifice, further wherein the airway comprises a first orifice at a first end of the second strip, and the at least two channels between the second strip and the film are in fluid communication with the first orifice at the first end of the second strip.

25. An airway according to claim 24, wherein the first orifice at the first end of the first strip is separated from the first orifice at the first end of the second strip by the film.

26. An airway according to any preceding claim, wherein the at least one groove comprised in the first strip of material is formed by heat stamping, laser cutting, laser engraving or thermo-forming.

27. An airway according to any preceding claim, wherein the at least one groove comprised in the second strip of material is formed by heat stamping, laser cutting, laser engraving or thermo-forming.

28. Use of an airway according to any preceding claim with a negative pressure wound dressing.

29. A negative pressure wound dressing comprising the airway according to any of claims 1 to 27.

30. A method of manufacturing an airway comprising the steps:
a. Providing a first strip of material having a first surface comprising a plurality of grooves;
b. Arranging a film over the first surface of the first strip of material;
c. Adhering the film to the first surface of the first strip of material around its longitudinal edges to form a plurality of channels between the first strip and the film;
d. Providing a second strip of material having a first surface comprising a plurality of grooves;
e. Arranging the second strip of material such that the first surface of the second strip of material is arranged over the film; and
f. Adhering the first surface of the second strip of material to the film around its longitudinal edges to form a plurality of channels between the second strip of material and the film.

31. A method of using a wound dressing comprising the airway according to any of claims 1 to 27.
